SEQUENCE LISTING (65 sequences)

SEQ ID NO: 1 - DNA, 3008 bp, Artificial Sequence
  Synthetic Construct
ctcacaaagccgagtccagctggagccacctccagactctctggccagagcctcagagtggccaacagtccctggccagcggttgctctatccaggctaagggcacccactcccctggagattcctgaacctgggccaggaagagccgaactagacaagcgtctccaatccatcccgttttgtccccggggttgtctgcagtcccgttagtcccttccctggcctgttcgccccttagacctccacacaggtctccctctgtgtaggaattcaccagaccctgtcctagccacccgctcagttcccccttctacctaggccttttctagctagttggatgggggatgggttagggaggctgtggtccccgagactccaggtgggagctcacgggcaggtactccgcaaaggagctggaaggcaggtctggaaaactgtcccccagatttaggattctggacaacgtccatctgctcatgctttggtcccccacgcacctccccgcctcctaaattccccatccccacctttcttgctcctttctgttgcttcgtcctctctgccttgggtctaaaactccaggttatgcctctgcaaacaaccccctcccttctaaccccagcagaactccgaggaaaggggcccgatgctccccccccttcccgcctgtggttagagggggcagtgtggcagtcccaagtgggggcgaccggagctgtctcggtgccccgcccgatcaggccactgggcacattgggggcgggaagctgggctcacgaaaggggcgactggccttggcaggtgtgggctctggtccggcctgggcgggctccgggggcggggtctcaggttacagccccgcggggggctagggggcggcccgcggtttgggccggtttgccagcctttggagcgaccgggagcatataactggagcctctgaagggggaagacgcagagcgccgctgggctgccgggtctcctgcctcctcctgctcctagggcctcctgcatgagggagcggtagagaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccac

SEQ ID NO: 2 - DNA, 728 bp, Artificial Sequence
  Synthetic Construct
gtttgtgattcagatgcctgtctgaggatctgcctcatagccctgctgcctggccagactgcctttgttctcttccataacttcgtatagcatacattatacgaagttatcttctgcacactaccactggcttggtgaaataatcagcaagccaccgatgggatctaatgagtgctctcaggccacccctgtcccatggagctcatactgaccctcacctgctctccttggctgcctttctaacagtatttcaatgggtcattgtcgtagatggcttcttcaggtagcattttcagttcactaacatactctgtttttacagctgacctgatggagttggacatggccatggagccggacagaaaagctgctgtcagccactggcagcagcagtcttacttggattctggaatccattctggtgccaccaccacagctccttccctgagtggcaagggcaaccctgaggaagaagatgttgacacctcccaagtcctttatgaatgggagcaaggcttttcccagtccttcacgcaagagcaagtagctggtaaagcatttgtgtttggaagataacttcgtatagcatacattatacgaagttatctagcattaagtttcttgacagggctgtgtgacagctcagccacagcacaagtgggttgaaggaagggcggagggtagcggagtcccacatgcctagtgagtgttggatttacctttttcag

SEQ ID NO: 3 - DNA, 232 bp, Artificial Sequence
  Synthetic Construct
gtttgtgattcagatgcctgtctgaggatctgcctcatagccctgctgcctggccagactgcctttgttctcttccataacttcgtatagcatacattatacgaagttatctagcattaagtttcttgacagggctgtgtgacagctcagccacagcacaagtgggttgaaggaagggcggagggtagcggagtcccacatgcctagtgagtgttggatttacctttttcag

SEQ ID NO: 4 - RNA, 21 nt, Mus musculus
aaggcuuuucccaguccuuca

SEQ ID NO: 5 - RNA, 21 nt, Mus musculus
aagaugauggugugccaagug

SEQ ID NO: 6 - RNA, 19 nt, Mus musculus
cugcgacucuugcgggaau

SEQ ID NO: 7 - RNA, 19 nt, Mus musculus
ucaaauggucccaaacgaa

SEQ ID NO: 8 - RNA, 19 nt, Mus musculus
agacuauggacaaagauua

SEQ ID NO: 9 - RNA, 19 nt, Mus musculus
ggcccauacaguuugaaua

SEQ ID NO: 10 - DNA, 20 bp, Artificial Sequence
  Synthetic Construct
agggtacctgaagctcagcg

SEQ ID NO: 11 - DNA, 20 bp, Artificial Sequence
  Synthetic Construct
cagtggctgacagcagcttt

SEQ ID NO: 12 - DNA, 20 bp, Artificial Sequence
  Synthetic Construct
tgaatcagctggcttttgtg

SEQ ID NO: 13 - DNA, 20 bp, Artificial Sequence
  Synthetic Construct
accttccagtaggggcaact

SEQ ID NO: 14 - DNA, 19 bp, Artificial Sequence
  Synthetic Construct
tttgagaacacggggaaga

SEQ ID NO: 15 - DNA, 20 bp, Artificial Sequence
  Synthetic Construct
actttgttgccaattccagg

SEQ ID NO: 16 - DNA, 20 bp, Artificial Sequence
  Synthetic Construct
aggacctggtagtgcgagtg

SEQ ID NO: 17 - DNA, 15 bp, Artificial Sequence
  Synthetic Construct
gcgtgtggcgaagaa

SEQ ID NO: 18 - DNA, 20 bp, Artificial Sequence
  Synthetic Construct
gcttttctcgtttgtggagc

SEQ ID NO: 19 - DNA, 20 bp, Artificial Sequence
  Synthetic Construct
tggaagtggcccatttagag

SEQ ID NO: 20 - DNA, 20 bp, Artificial Sequence
  Synthetic Construct
gaggaggaggaagagcagag

SEQ ID NO: 21 - DNA, 20 bp, Artificial Sequence
  Synthetic Construct
tgggatgttctccagatgtt

SEQ ID NO: 22 - DNA, 20 bp, Artificial Sequence
  Synthetic Construct
ctcagaagaaggttggctgg

SEQ ID NO: 23 - DNA, 20 bp, Artificial Sequence
  Synthetic Construct
acctcgctcaccttgaagaa

SEQ ID NO: 24 - DNA, 22 bp, Artificial Sequence
  Synthetic Construct
tccttcctgctgaagaagacca

SEQ ID NO: 25 - DNA, 22 bp, Artificial Sequence
  Synthetic Construct
taaagctggtgatggtgttggc

SEQ ID NO: 26 - DNA, 20 bp, Artificial Sequence
  Synthetic Construct
gcatggccaagaagacatcc

SEQ ID NO: 27 - DNA, 19 bp, Artificial Sequence
  Synthetic Construct
cctcgggtttccacgtctc

SEQ ID NO: 28 - DNA, 20 bp, Artificial Sequence
  Synthetic Construct
ccacaccaaattcctgttca

SEQ ID NO: 29 - DNA, 20 bp, Artificial Sequence
  Synthetic Construct
actggtaagtggggcaagac

SEQ ID NO: 30 - DNA, 20 bp, Artificial Sequence
  Synthetic Construct
accccaaggacctaaaggaa

SEQ ID NO: 31 - DNA, 20 bp, Artificial Sequence
  Synthetic Construct
ccccaggataccctgttttt

SEQ ID NO: 32 - DNA, 18 bp, Artificial Sequence
  Synthetic Construct
agggaggatcaagtcccg

SEQ ID NO: 33 - DNA, 18 bp, Artificial Sequence
  Synthetic Construct
gaacagactccggcgcta

SEQ ID NO: 34 - DNA, 22 bp, Artificial Sequence
  Synthetic Construct
tcctgaccaaaaacctcaaagg

SEQ ID NO: 35 - DNA, 19 bp, Artificial Sequence
  Synthetic Construct
tcgttcatgcagagcctgc

SEQ ID NO: 36 - DNA, 20 bp, Artificial Sequence
  Synthetic Construct
ccttgctgctcaacctccac

SEQ ID NO: 37 - DNA, 20 bp, Artificial Sequence
  Synthetic Construct
cacacaggatggcttgaaga

SEQ ID NO: 38 - DNA, 24 bp, Artificial Sequence
  Synthetic Construct
acagcgttcatcttcgcaatcacc

SEQ ID NO: 39 - DNA, 24 bp, Artificial Sequence
  Synthetic Construct
aaatcgatgttgtcactgcagccc

SEQ ID NO: 40 - DNA, 20 bp, Artificial Sequence
  Synthetic Construct
ggctcctctcggatacctct

SEQ ID NO: 41 - DNA, 20 bp, Artificial Sequence
  Synthetic Construct
gggcatgatctccacgtagt

SEQ ID NO: 42 - DNA, 21 bp, Artificial Sequence
  Synthetic Construct
ctcaaaggcctgatccagagc

SEQ ID NO: 43 - DNA, 20 bp, Artificial Sequence
  Synthetic Construct
gtcccttgtgtcaccacctt

SEQ ID NO: 44 - DNA, 20 bp, Artificial Sequence
  Synthetic Construct
tgcatgatcccatgcccttt

SEQ ID NO: 45 - DNA, 20 bp, Artificial Sequence
  Synthetic Construct
accaaacagctgcaacacct

SEQ ID NO: 46 - DNA, 20 bp, Artificial Sequence
  Synthetic Construct
tacgtgcaagtgaatgcggt

SEQ ID NO: 47 - DNA, 20 bp, Artificial Sequence
  Synthetic Construct
tggttctttccctgtgagca

SEQ ID NO: 48 - DNA, 20 bp, Artificial Sequence
  Synthetic Construct
ttcctccacaacacatggca

SEQ ID NO: 49 - DNA, 20 bp, Artificial Sequence
  Synthetic Construct
atgcaaggcaagggcaaaca

SEQ ID NO: 50 - DNA, 20 bp, Artificial Sequence
  Synthetic Construct
tgctatggcatcaagtggct

SEQ ID NO: 51 - DNA, 22 bp, Artificial Sequence
  Synthetic Construct
ccagctgtttacagtgttgcgt

SEQ ID NO: 52 - DNA, 20 bp, Artifical Sequence
  Synthetic Construct
atccgcaagctgcaatgcta

SEQ ID NO: 53 - DNA, 20 bp, Artificial Sequence
  Synthetic Construct
tgtgcttgggaaaccggaaa

SEQ ID NO: 54 - DNA, 20 bp, Artificial Sequence
  Synthetic Construct
tggcctggttcaaggaaagt

SEQ ID NO: 55 - DNA, 20 bp, Artificial Sequence
  Synthetic Construct
tgcctttgagcttcagcgtt

SEQ ID NO: 56 - DNA, 20 bp, Artificial Sequence
  Synthetic Construct
tggaaagaacaagcgccact

SEQ ID NO: 57 - DNA, 20 bp, Artificial Sequence
  Synthetic Construct
tgcaaagccatccgcatcaa

SEQ ID NO: 58 - DNA, 22 bp, Artificial Sequence
  Synthetic Construct
tggtccgcgtaaagtatgggaa

SEQ ID NO: 59 - DNA, 22 bp, Artificial Sequence
  Synthetic Construct
ctgcattgccacccatggtaaa

SEQ ID NO: 60 - DNA, 22 bp, Artificial Sequence
  Synthetic Construct
tcagtggatcttcgcagttgga

SEQ ID NO: 61 - DNA, 22 bp, Artificial Sequence
  Synthetic Construct
acaggatgccttccttggatct

SEQ ID NO: 62 - DNA, 22 bp, Artificial Sequence
  Synthetic Construct
tctggctttaacgaggagcctt

SEQ ID NO: 63 - DNA, 22 bp, Artificial Sequence
  Synthetic Construct
ggcaagcagggttggaatcttt

SEQ ID NO: 64 - DNA, 20 bp, Artificial Sequence
  Synthetic Construct
tgcatggaggccatcatctt

SEQ ID NO: 65 - DNA, 20 bp, Artificial Sequence
  Synthetic Construct
tgcaaatggcagcaccaaca

(19) United States
(12) Patent Application Publication
(10) Pub. No.: US 2011/0289605 A1
(43) Pub. Date: Nov. 24, 2011

(54) Animal Model for Osteoarthritis and Intervertebral Disc Disease

(75) Inventor: Di Chen, Pittsford, NY (US)

(73) Assignee: THE UNIVERSITY OF ROCHESTER, Rochester, NY (US)

(21) Appl. No.: 13/130,490

(22) PCT Filed: Nov. 25, 2009
(86) PCT No.: PCT/US2009/065911

§ 371 Date: Aug. 4, 2011

Related U.S. Application Data
(60) Provisional application No. 61/117,766, filed on Nov. 25, 2008.
(60) Provisional application No. 61/231,852, filed on Aug. 6, 2009.

(51) Int. Cl. A01K 67/027; A61K 39/395; A61K 38/02; A61K 38/45; A61P 19/02; G01N 33/573; A61K 31/138; C12N 5/10; C12Q 1/02; C12Q 1/68; G01N 33/566; G01N 33/00; A61K 31/713; A61P 19/00

(52) U.S. Cl. 800/3; 514/44 A; 424/172.1; 514/16.7; 514/16.8; 424/94.5; 800/9; 514/648; 435/325; 435/29; 435/6.12; 435/7.1; 435/6.13; 435/7.4

(57) ABSTRACT

Provided herein is a transgenic animal whose genome comprises a first nucleic acid sequence encoding a fusion polypeptide, wherein the fusion polypeptide comprises a Cre recombinase and a mutated ligand binding domain of human estrogen receptor (CreER), wherein the first nucleic acid is operably linked to a chondrocyte-specific promoter and a second nucleic acid sequence encoding a β-catenin polypeptide, wherein the second nucleic acid sequence comprises one or more loxP sequences. Also provided is a method of modifying a transgenic animal comprising administering tamoxifen to the transgenic animal. Also provided are methods of screening for an agent that reduces or prevents Cre-Negative Control one or more symptoms of osteoarthritis or intervertebral disc disease in a subject. Methods for identifying a subject with or at risk of developing osteoarthritis or intervertebral disc disease are also provided, as well as methods of treating or preventing osteoarthritis or intervertebral disc disease in a subject.

### CROSS REFERENCE TO RELATED APPLICATIONS

This application claims the benefit of U.S. Provisional Application No. 61/117,766, filed on Nov. 25, 2008, and U.S. Provisional Application No. 61/231,852, filed on Aug. 6, 2009, which are incorporated by reference herein in their entireties.

### STATEMENT REGARDING FEDERALLY FUNDED RESEARCH

This invention was made with government funding under Grant Nos. RO1 AR051189, RO1 AR054465, and KO2 AR052411 from the National Institutes of Health. The government has certain rights in this invention.

### BACKGROUND

Arthritis is the number one cause of disability in the United States. Osteoarthritis (OA), the most common form of arthritis, is a non-inflammatory degenerative joint disease characterized by dysfunction of articular chondrocytes, articular cartilage degradation, osteophyte formation, and subchondral sclerosis. OA affects nearly 21 million people in the United States. It is estimated that 80% of the population will have radiographic evidence of OA by age 65, although only 60% of those will be symptomatic. The progression of OA is slow and eventually results in destruction and total loss of articular cartilage of various joints, including fingers, knees, hips, and spine. The disease process leads to limitation of joint movement, joint deformity, joint stiffness, inflammation, and severe pain. While there are several strategies to reduce symptoms and/or decelerate disease progression, there are few therapeutic approaches for OA patients. Treatments for OA include non-steroidal anti-inflammatory drugs and local injections of glucocorticoid, and in severe cases, joint replacement surgery. Currently, there is limited information about the cellular and/or molecular events that occur during articular cartilage degeneration.

Osteoarthritis (OA) mainly involves dysfunction of articular chondrocytes, the only cell type present in articular cartilage. Articular chondrocytes produce and maintain the extracellular matrix, which is responsible for providing the appropriate structure and function of the cartilagenous tissue. The function of articular chondrocytes is regulated by a variety of growth factors, including Wnt family members. β-catenin is a key molecule in the canonical Wnt signaling pathway and plays a critical role in multiple steps during chondrocyte formation and maturation. Genetic evidence is critical for understanding the role of β-catenin in skeletal development. However, this is limited by the embryonic or immediate postnatal lethality of β-catenin gene deletion and activation.

Disc degeneration is expressed by the production of abnormal components of the matrix or by an increase in the mediators of matrix degradation. In degenerative discs, cells in the annulus and nucleus aggregate and form colonies, which is accompanied by a decrease in the content of type II collagen and an increase in type I collagen. In addition, expression of colX and other hypertrophic chondrocyte marker genes is also increased in the annulus and nucleus areas. Further, MMP13 expression is increased in degenerative rat discs. Disc degeneration is influenced by many factors including genetic factors, age, nutrition, and mechanical signals. However, very little is known about the signaling mechanism that controls changes in cell phenotype and gene expression during disc degeneration.

### SUMMARY

Provided are transgenic animal models for osteoarthritis or intervertebral disc disease. Specifically provided are transgenic animals whose genome comprises a first nucleic acid sequence encoding a fusion polypeptide, wherein the fusion polypeptide comprises a Cre recombinase and a mutated ligand binding domain of human estrogen receptor (CreER) operably linked to a chondrocyte-specific promoter and a second nucleic acid sequence encoding a β-catenin polypeptide, with the second nucleic acid sequence comprising one or more loxP sequences. The transgenic animal can, for example, be a mouse.

Also provided are progeny animals resulting from a cross between a first transgenic animal whose genome comprises a first nucleic acid sequence encoding a fusion polypeptide, wherein the fusion polypeptide comprises a Cre recombinase and a mutated ligand binding domain of human estrogen receptor (CreER) operably linked to a chondrocyte-specific promoter and a second transgenic animal whose genome comprises a second nucleic acid sequence encoding a β-catenin polypeptide, with the second nucleic acid sequence comprising one or more loxP sequences. The progeny animal can, for example, be a mouse.

Also provided are methods to modify a transgenic animal. The methods comprise administering tamoxifen to the transgenic animal whose genome comprises a first nucleic acid sequence encoding a fusion polypeptide, wherein the fusion polypeptide comprises a Cre recombinase and a mutated ligand binding domain of human estrogen receptor (CreER) operably linked to a chondrocyte-specific promoter and a second nucleic acid sequence encoding a β-catenin polypeptide, wherein the second nucleic acid sequence comprises two loxP sequences. The first loxP sequence is located 5′ to the third exon of the second nucleic acid sequence, and the second loxP sequence is located 3′ to the third exon of the second nucleic acid sequence. Administration of tamoxifen results in the deletion of the third axon of the second nucleic acid sequence. The deletion of the third exon of the second nucleic acid sequence results in a third nucleic acid sequence, wherein the third nucleic acid sequence encodes a β-catenin fusion polypeptide lacking the amino acids encoded by the third exon.

Further provided are methods of screening an agent that reduces or prevents one or more symptoms of osteoarthritis or intervertebral disc disease. The methods comprise providing a transgenic animal or a cell whose genome comprises a first nucleic acid sequence encoding a fusion polypeptide, wherein the fusion polypeptide comprises a Cre recombinase and a mutated ligand binding domain of human estrogen receptor (CreER) operably linked to a chondrocyte-specific promoter, and a second nucleic acid sequence comprising a β-catenin fusion polypeptide; contacting the transgenic animal with an agent to be screened; and determining whether the agent reduces or prevents one or more symptoms of osteoarthritis or intervertebral disc disease.

Further provided are methods of identifying a subject with or at risk for developing osteoarthritis or intervertebral disc disease. The methods comprise obtaining a biological sample from the subject and determining a level of expression or activity of β-catenin in the sample. An increase in β-catenin expression or activity as compared to a control indicates the subject has or is at risk for developing osteoarthritis or intervertebral disc disease.

Further provided are methods of treating or preventing osteoarthritis or intervertebral disc disease in a subject. The methods comprise selecting a subject with or at risk of developing osteoarthritis or intervertebral disc disease and administering to the subject an effective amount of a first therapeutic agent comprising a β-catenin inhibitor or MMP-13 inhibitor. The methods further comprise administering one or more second therapeutic agents to the subject.

### DESCRIPTION OF DRAWINGS

FIGS. 1A and 1B show Tamoxifen (TM)-induced Cre-recombination in adult articular chondrocytes. FIG. 1A shows histological sections demonstrating 84% recombination efficiency in 5-month-old Col2a1-CreER^{T2};R26R mice (n=3) as compared to a Cre-negative control. FIG. 1B shows histological sections demonstrating 76% recombination efficiency in 8-month-old Col2a1-CreER^{T2};R26R mice (n=3) as compared to a Cre-negative control.

FIG. 2 shows histological sections demonstrating increased β-catenin protein levels in articular chondrocytes from p-catenin cAct mice in comparison with Cre-negative control mice.

FIGS. 3A-3C show 5-month-old β-catenin cAct mice developed a mild OA-like phenotype. FIG. 3A shows histological sections demonstrating reduced Safranin O/Fast green staining in β-catenin cAct mice compared to Cre-negative control mice. FIG. 3B shows histological sections demonstrating reduced Alcian blue/Hematoxylin & orange G staining in β-catenin cAct mice compared to Cre-negative control mice. FIG. 3C shows a histogram representing histomorphometric analysis that demonstrated there is 38% reduction in auricular cartilage area in β-catenin cAct mice (n=4). *p<0.05, unpaired Student's t-test.

FIGS. 4A-4J show 8-month-old β-catenin cAct mice develop a severe OA-like phenotype. FIG. 4A shows histological sections demonstrating reduced levels of Safranin O/Fast green staining in 8-month-old β-catenin cAct compared to Cre-negative control mice. FIG. 4B shows histological sections demonstrating reduced levels of Alcian blue/Hematoxylin & orange G staining in 8-month old β-catenin cAct mice compared to Cre-negative control mice. FIG. 4C shows a higher magnification of Alcian blue/Hematoxylin & orange G-stained section of FIG. 4B demonstrating cell cloning in 8-month old β-catenin cAct mice compared to Cre-negative control mice. FIG. 4D shows X-ray radiography demonstrating osteophyte formation in β-catenin cAct mice. FIGS. 4E-J show high magnification pictures of Safranin O/Fast green and Alcian blue/Hematoxylin & orange G staining FIG. 4E shows formation of chondrophytes. FIG. 4F shows loss of the entire articular cartilage layer. FIG. 4G shows formation of chondrophytes. FIG. 4H shows formation of chondrophytes and cell cloning. FIG. 4I shows formation of chondrophytes. FIG. 4J shows formation of clefts and new woven bone formation in knee joints from 8-month old β-catenin cAct mice.

FIGS. 5A-5G show chondrocyte differentiation is accelerated in β-catenin conditional activation (cAct) mice. FIG. 5A shows type I collagen (col1) and type II collagen (col2) expression in isolated primary articular chondrocytes from β-catenin cAct mice and Cre-negative control mice (n=10) demonstrating minimal fibroblast or osteoblast contamination. FIG. 513 shows a histogram demonstrating Bmp2 expression is increased 6-fold and greater than 2-fold increases in expression of Bmp6 and Gdf5 are observed in β-catenin cAct mice. FIG. 5C shows a histogram demonstrating aggrecan, Mmp-9, and Mmp- 13 expression is increased 2.5, 4, and 3.5-fold, respectively. FIG. 5D shows a histogram demonstrating Alp, osteocalcin (Oc), and type X collagen (colX) expression is increased 2.5, 3, and 3.5-fold, respectively. FIG. 5E shows a histogram demonstrating that colX, Mmp-9, and Mmp-13 expression is increased 3, 2, and 3-fold, respectively, in articular tissues from 2-month-old β-catenin cAct mice. FIG. 5F shows a histogram demonstrating that Bmp2 expression is increased 5-fold in articular tissues derived from β-catenin cAct mice. *p<0.05, unpaired Student's t-test. FIG. 5G shows histological sections demonstrating an increase in cellular MMP-13 protein expression in β-catenin cAct mice compared to Cre-negative mice.

FIGS. 6A-6I show activation of β-catenin signaling alters the expression of Wnt ligands, Wnt antagonists, and Wnt target genes. FIGS. 6A, 6B, and 6D show histograms demonstrating the expression of Wnt1, Wnt4, and Wnt7 a was decreased 70-90% in primary articular chondrocytes isolated from 1-month-old β-catenin cAct mice compared to Cre-negative control mice (n=8). FIGS. 6C and 6E show histograms demonstrating no significant change was found in the expression of Wnt4 and Wnt7b . FIGS. 6F and 6G show histograms demonstrating the expression of Wnt5 and Wnt11 was increased 1.7 and 2.4-fold, respectively. FIG. 6H shows a histogram demonstrating the expression of sFRP2 (Wnt antagonist) was also increased 2.3-fold. FIG. 61 shows a histogram demonstrating that expression of WISP1 (Wnt target gene) was increased 2.6-fold.

FIGS. 7A-7C show β-catenin levels are increased in human OA subjects. FIG. 7A shows β-catenin immunostaining of normal human joints demonstrating low cellular β-catenin expression (n=20). FIG. 7B shows β-catenin immunostaining with low Mankin grade OA cartilage from knee arthroplasty patients demonstrating increased cellular β-catenin expression (n=9). FIG. 7C shows β-catenin immunostaining with high Mankin grade OA cartilage from knee arthroplasty patients demonstrating increased β-catenin expression (n=13).

FIG. 8 shows high efficiency of Cre-recombination in intervertebral disc (IVD) cells of Col2a1-CreER^{T2} transgenic mice. To determine Cre-recombination efficiency in IVD tissue, Col2a1-CreER^{T2} transgenic mice were bred with Rosa26 reporter mice (R26R strain). TM was administered to 2-week-old Col2a1-CreER^{T2};R26R transgenic mice and X-Gal staining was performed when mice were at 1 month of age. High Cre-recombination efficiency was observed in annulus fibrosus (AF) cells and endplate cartilage (EC) cells but not nucleus pulposus (NP) cells.

FIG. 9 shows overexpression of β-catenin protein in IVD cells of β-catenin conditional activation (cAct) mice. Col2a1-CreER^{T2} transgenic mice were bred with β-catenin^{fx(Ex3)/fx(Ex3)} mice. TM was administered to 2-week-old mice resulting in Col2a1-CreER^{T2};β-catenin^{fx(Ex3)/wt} mice. Cre-negative littermates were used as negative controls and were treated with TM under the same condition. Mice were sacrificed at 1 month for immunostaining. β-catenin protein expression was significantly up regulated in β-catenin cAct mice, especially in annulus fibrosus cells (indicated by arrows).

FIG. 10 shows the loss of endplate cartilage in β-catenin cAct mice. TM was administered into 2-week-old Col2a1-CreER^{T2};β-catenin^{fx(Ex3)/wt} mice. Cre-negative littermates were used as negative controls and were treated with TM under the same condition. Mice were sacrificed at 1 month for micro-CT analysis. Osteophyte formation (grey arrows) and loss of endplate cartilage (white arrows, lower panels) were observed in β-catenin cAct mice but not in Cre-negative littermate controls.

FIGS. 11A-11E show the destruction of IVD tissue in β-catenin cAct mice. TM was administered into 2-week-old Col2a1-CreER^{T2};β-catenin^{fx(Ex3)/wt} mice. Cre-negative littermates were used as negative controls and were treated with TM under the same condition (FIGS. 11A and 11D). Mice were sacrificed at 1 month for histological analysis. Loss of endplate cartilage (FIGS. 11B and 11C), formation of new blood vessels and new woven bone and disorganized annulus fibrosus cells (FIGS. 11B and 11C), chondrophyte formation (FIG. 11E) and reduced endplate cartilage area (FIG. 11E) were observed in β-catenin cAct mice but not in Cre-negative littermate controls.

FIGS. 12A-12G show histograms demonstrating the alteration of gene expression in IVD tissue of β-catenin cAct mice. TM was administered into 2-week-old Col2a1-CreER^{T2};β-catenin^{fx(Ex3)/wt} mice. Cre-negative littermates were used as negative controls and were treated with TM under the same condition. Mice were sacrificed at 3 weeks of age and primary disc cells were isolated from β-catenin cAct mice and Cre-negative control mice. Total RNA was extracted from primary disc cells and gene expression was analyzed by real-time PCR. Expression of Mmp-13 (FIG. 12C) but not Mmp-2 (FIG. 12A) and Mmp-3 (FIG. 12B) was significantly increased in disc cells derived from β-catenin cAct mice. Expression of type IX collagen (Col-9) (FIG. 12D) was significantly decreased and expression of type X collagen (Col-X) (FIG. 12E) was significantly increased in β-catenin cAct mice. In contrast, a significant increase in expression of Adamts4 (FIG. 12F) and Adamts5 (FIG. 12G) was also detected in β-catenin cAct disc cells.

FIG. 13 shows changes in MMP-13 protein expression in β-catenin cAct mice. TM was administered into 2-week-old Col2a1-CreER^{T2};β-catenin^{fx(Ex3)/wt} mice. Cre-negative littermates were used as negative controls and were treated with TM under the same condition. Mice were sacrificed at 1 month and immunostaining was performed. Expression of MMP-13 protein was significantly increased in disc cells of β-catenin cAct mice.

FIGS. 14A and 14B show reduction of the length of spine in 3-month-old β-catenin cAct mice. X-ray radiographic analysis showed that lengths of spine were significantly decreased in β-catenin cAct mice compared to Cre-negative littermate controls. FIG. 14A shows a representative image of the full mouse comparing the Cre-negative control to the β-catenin cAct mouse. FIG. 14B shows a representative image of the spinal column comparing the Cre-negative control to the β-catenin cAct mouse.

FIGS. 15A and 15B show severe osteophyte formation and disc space narrowing in 3-month-old β-catenin cAct mice. TM was administered into 2-week-old Col2a1-CreER^{T2};β-catenin^{fx(Ex3)/wt} mice. Cre-negative littermates were used as negative controls and were treated with TM under the same condition. Mice were sacrificed at 3 months for micro-CT analysis. Massive amounts of osteophyte (light grey arrows) and disc space narrowing (dark grey arrows) were observed in β-catenin cAct mice but not in Cre-negative littermate controls. FIG. 15A shows an image of the coronary view comparing the spine of the Cre-negative control to the β-catenin cAct mouse. FIG. 15B shows an image of the lateral view comparing the spine of the Cre-negative control to the β-catenin cAct mouse.

FIGS. 16A and 16B show severe disc destruction phenotype in β-catenin cAct mice. TM was administered into 2-week-old Col2a1-CreER^{T2};β-catenin^{fx(Ex3)/wt} mice. Cre-negative littermates were used as negative controls and were treated with TM under the same condition. Mice were sacrificed at 3 months for histological analysis. Severe loss of proteoglycan, demonstrated by reduced Alcian blue (FIG. 16A) and Safranin O (FIG. 16B) staining, loss of endplate cartilage and disorganized annulus fibrosus cells were observed in β-catenin cAct mice but not in Cre-negative littermate controls.

FIG. 17 shows the rescue of disc destruction phenotype by deletion of the Mmp-13 gene under β-catenin cAct background. TM was administered into 2-week-old Col2a1-CreER^{T2};β-catenin^{fx(Ex3)/wt} and Col2a1-CreER^{T2};β-catenin^{(Ex3)/wt};Mmp13^{fx/fx} mice. Cre-negative littermates were used as negative controls and were treated with tamoxifen under the same condition. Mice were sacrificed at 1 and 3 months for micro-CT analysis. Loss of endplate cartilage (grey arrows) and disc space narrowing (white arrows) were observed in 1- and 3-month-old β-catenin cAct mice (middle panel). Deletion of the Mmp-13 gene significantly reversed the loss of endplate cartilage and disc space narrowing phenotypes observed in β-catenin cAct mice (right panel).

FIG. 18 shows the rescue of disc destruction phenotype by deletion of the Mmp-13 gene under β-catenin cAct background. TM was administered into 2-week-old Col2a1-CreER^{T2};β-Catenin^{fx(Ex3)/wt} and Col2a1-CreER^{T2};β-catenin^{fx(Ex3)/wt};Mmp13^{fx/fx} mice. Cre-negative littermates were used as negative controls and were treated with TM under the same condition. Mice were sacrificed at 1 and 3 months for histological analysis. Loss of endplate cartilage, reduced proteoglycan protein levels and disorganized annulus fibrosus cells were observed in 1- and 3-month-old β-catenin cAct mice (middle panel). Deletion of the Mmp-13 gene significantly reversed the loss of endplate cartilage and reduced proteoglycan protein levels (demonstrated by Alcian blue staining) observed in β-catenin cAct mice (right panel).

FIGS. 19A-19C show Wnt3a induces Mmp-13 and Runx2 expression. FIG. 19A shows a histogram demonstrating that Wnt3a stimulated Mmp-13 expression. FIG. 19B shows an image of a Western blot demonstrating that Wnt3a stimulated Runx2 protein expression in a time-dependent manner. FIG. 19C shows a histogram demonstrating that both Runx2 and Wnt3a stimulated Mmp-13 promoter activity and mutation of the Runx2 binding site completely abolished Runx2 or Wnt3a-induced Mmp-13 promoter activity.

### DETAILED DESCRIPTION

Provided herein is a transgenic animal whose genome comprises a first nucleic acid sequence encoding a fusion polypeptide, wherein the fusion polypeptide comprises a Cre recombinase and a mutated ligand binding domain of human estrogen receptor (CreER) operably linked to a chondrocyte-specific promoter and a second nucleic acid sequence encoding a β-catenin polypeptide, wherein the second nucleic acid sequence comprises one or more loxP sequences. Optionally, the chondrocyte-specific promoter is selected from the group consisting of a Col2a1 promoter, a fgfr-3 promoter, an aggrecan promoter, and a Col11a2 promoter. Optionally, the chondrocyte-specific promoter is Col2a1. Optionally, the second nucleic acid sequence comprises two loxP sequences. Optionally the second nucleic acid sequence further comprises at least a first exon, a second exon, and a third exon. Optionally, the second nucleic acid comprises a first loxP sequence located 5′ to the third exon of the second nucleic acid sequence and a second loxP sequence located 3′ to the third exon of the second nucleic acid sequence. Optionally the transgenic animal comprises a first nucleic acid sequence comprising SEQ ID NO:1. Optionally, the transgenic animal comprises a second nucleic acid sequence comprising SEQ ID NO:2. Optionally, the transgenic animal is a mouse.

Also provided herein is an isolated cell of the transgenic animal whose genome comprises a first nucleic acid sequence encoding a fusion polypeptide, wherein the fusion polypeptide comprises a Cre recombinase and a mutated ligand binding domain of human estrogen receptor (CreER). The first nucleic acid is operably linked to a chondrocyte-specific promoter. The cell further comprises a second nucleic acid sequence encoding a β-catenin polypeptide, wherein the second nucleic acid sequence comprises one or more loxP sequences. Optionally, the isolated cell is a chondrocyte or a fibroblast (e.g., an intervertebral disc cell), but other cell types are useful herein.

Also provided herein is a progeny animal resulting form a cross between two transgenic animals. The first transgenic animal's genome comprises a first nucleic acid sequence encoding a fusion polypeptide, wherein the fusion polypeptide comprises a Cre recombinase and a mutated ligand binding domain of human estrogen receptor (CreER). The first nucleic acid is operably linked to a chondrocyte-specific promoter. The second transgenic animal's genome comprises a second nucleic acid sequence encoding a β-catenin polypeptide, wherein the second nucleic acid sequence comprises one or more loxP sequences. Optionally, the chondrocyte-specific promoter of the progeny animal is selected from the group consisting of the Col2a1 promoter, a fgfr-3 promoter, an aggrecan promoter, and a Col11a2 promoter. Optionally, the second nucleic acid sequence of the progeny animal comprises two loxP sequences. Optionally, the second nucleic acid sequence of the progeny animal further comprises at least a first exon, a second exon, and a third exon. Optionally, the second nucleic acid sequence of the progeny animal comprises a first loxP sequence located 5′ to the third exon of the second nucleic acid sequence and a second loxP sequence located 3′ to the third exon of the second nucleic acid sequence. Optionally, the first nucleic acid sequence of the progeny animal comprises SEQ ID NO:1. Optionally, the second nucleic acid sequence of the progeny animal comprises SEQ ID NO:2. Optionally, the progeny animal is a mouse.

Also provided herein is an isolated cell of the progeny animal resulting from a cross between a first and second transgenic animals. Optionally, the isolated cell is a chondrocyte or a fibroblast, but other cell types are useful herein.

Provided herein are methods of modifying a transgenic animal. The methods comprise administering tamoxifen to the transgenic animal whose genome comprises a first nucleic acid sequence encoding a fusion polypeptide, wherein the fusion polypeptide comprises a Cre recombinase and a mutated ligand binding domain of human estrogen receptor (CreER). The first nucleic acid is operably linked to a chondrocyte-specific promoter. The genome of the transgenic animal further comprises a second nucleic acid sequence encoding a β-catenin polypeptide, wherein the second nucleic acid sequence comprises two loxP sequences. The first loxP sequence is located 5′ to the third exon of the second nucleic acid sequence and the second loxP sequence is located 3′ to the third exon of the second nucleic acid sequence. Administration of tamoxifen results in the deletion of the third exon of the second nucleic acid sequence. The deletion of the third exon of the second nucleic acid sequence results in a third nucleic acid sequence, wherein the third nucleic acid sequence encodes a β-catenin fusion polypeptide lacking the amino acids encoded by the third exon. Optionally, the tamoxifen is 4-hydroxy tamoxifen, which is an active metabolite of tamoxifen.

Also provided herein is a transgenic animal made by the aforementioned method of modifying a transgenic animal comprising administering tamoxifen to the transgenic animal. Optionally, the third nucleic acid sequence of the modified transgenic animal comprises SEQ ID NO:3. Also provided herein is an isolated cell of the modified transgenic animal. Optionally, the isolated cell of the modified transgenic animal is a chondrocyte or a fibroblast.

Optionally, the transgenic animals described above can be crossed with other transgenic animal models of development and/or disease (e.g., Mmp13^{fx/fx} as described in Example 8). Also provided herein are progeny animals resulting from a cross between a transgenic animal whose genome comprises a first nucleic acid sequence encoding a fusion polypeptide, wherein the fusion polypeptide comprises a Cre recombinase and a mutated ligand binding domain of human estrogen receptor (CreER) operably linked to a chondrocyte-specific promoter and a second nucleic acid sequence encoding a β-catenin polypeptide, wherein the second nucleic acid sequence comprises one or more loxP sequences and another transgenic animal model of development and/or disease. Also provided are methods of modifying the progeny animals produced. The methods can, for example, comprise administering tamoxifen to the progeny animal. Further provided is an isolated cell from the modified progeny animals. Optionally, the isolated cell of the modified progeny animal is a chondrocyte or a fibroblast.

Transgenic animals are useful in the study of OA and intervertebral disc disease. For example, as shown herein, conditional activation of the β-catenin gene in articular chondrocytes in adult mice leads to OA-like articular cartilage destruction associated with accelerated chondrocyte differentiation, showing that β-catenin signaling plays a critical role in OA pathogenesis. β-catenin cAct mice show spontaneous OA lesion in articular cartilage, demonstrating that β-catenin plays a role in OA development caused by Frzb mutations or other mechanisms which lead to activation of β-catenin signaling.

Also shown herein, mRNA expression of Bmp2 was significantly increased in articular chondrocytes and articular cartilage tissues (5 to 6-fold increase) derived from β-catenin cAct mice. Gene expression analysis also showed that expression of chondrocyte differentiation marker genes, regulated by BMP-2 such as Alp, Oc, and colX, were also significantly increased in articular chondrocytes derived from β-catenin cAct mice. BMP-2 induces de novo osteophyte formation in the normal murine knee joint. As demonstrated herein, the expression of Mmp-13 mRNA was increased in articular chondrocytes and intervertebral disc cells derived from β-catenin cAct mice. MMP-13 is a potent enzyme which degrades cartilage matrix with preference for type II collagen and the expression of MMP-13 is up regulated in human OA knee joints. The transgenic mice expressing constitutively active Mmp-13 show changes in the OA-like phenotype, suggesting a close relationship between Mmp-13 and cartilage destruction in OA.

As shown herein, to determine changes in Wnt signaling, expression of Wnt ligands and Wnt antagonists in articular chondrocytes in which β-catenin signaling is activated was examined. Wnt1, Wnt3a, Wnt4, Wnt7a and Wnt7b are involved in canonical Wnt signaling, and Wnt5 and Wnt11 are involved in non-canonical Wnt signaling. As shown herein, expression of Wnt1, Wnt3a and Wnt7a was significantly reduced and expression of sFRP2 was significantly increased, showing negative feedback regulation in genes involved in canonical Wnt signaling. In contrast, expression of Wnt5 and Wnt11 was significantly increased, showing that activation of β-catenin signaling up-regulates non-canonical Wnt signaling in articular chondrocytes.

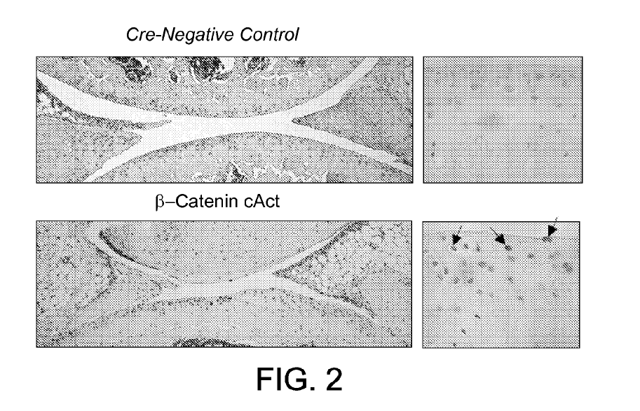

The mechanism underlying β-catenin-induced OA is that β-catenin promotes articular chondrocyte maturation. As shown herein in FIG. 2, the β-catenin positive cells in the resting zone have lost their flattened phenotype, showing that these cells are undergoing maturation as a result of increased β-catenin within the cells. In addition, β-catenin-positive cells are closer to the articular surface.

Selective inhibition of β-catenin signaling in chondrocytes causes delay of growth plate chondrocyte maturation and articular cartilage destruction in Col2a1-ICAT transgenic mice. Furthermore, cell apoptosis of articular chondrocytes is significantly increased in these transgenic mice. Thus, β-catenin signaling plays a critical role in prevention of articular chondrocytes from undergoing apoptosis under normal physiological conditions.

As shown herein, conditional activation of the β-catenin gene in articular chondrocytes in adult mice leads to premature chondrocyte differentiation and the development of an OA-like phenotype. Data provided herein have provided novel and definitive evidence about the role of β-catenin signaling in articular chondrocyte function and OA pathogenesis.

Similarly, intervertebral disc cells (annulus fibrous cells, endplate cartilage cells) of the β-catenin cAct transgenic mice showed overexpression of β-catenin protein, increased levels of Mmp-13 and other genes, and destruction of IVD tissue. The disc destruction in these transgenic mice was phenotypically reversed with the deletion of the Mmp-13 gene in transgenic mice produced by crossing a β-catenin cAct mouse with a Mmp-13 conditional knockout mouse. The conditional knockout of Mmp-13 resulted in the entire disc tissue morphology returned to normal and proteoglycan proteins levels were increased and loss of endplate cartilage was restored.

Provided herein is a method of screening for an agent that reduces or prevents one or more symptoms of osteoarthritis or intervertebral disc disease comprising the steps of: (a) providing a transgenic animal whose genome comprises (i) a first nucleic acid sequence encoding a fusion polypeptide, wherein the fusion polypeptide comprises a Cre recombinase and a mutated ligand binding domain of human estrogen receptor (CreER), wherein the first nucleic acid is operably linked to a chondrocyte-specific promoter and (ii) a second nucleic acid sequence comprising a β-catenin fusion polypeptide; (b) contacting the transgenic animal with an agent to be tested; and (c) determining whether the agent reduces or prevents one or more symptoms of osteoarthritis or intervertebral disc disease. Determining whether the agent reduces or prevents one or more symptoms of osteoarthritis or intervertebral disc disease can include, for example, determining the level of expression of the β-catenin fusion polypeptide. A decrease in the level of expression of the β-catenin fusion polypeptide as compared to a control indicates the agent reduces or prevents one or more symptoms of osteoarthritis or intervertebral disc disease. Determining whether the agent reduces or prevents one or more symptoms of osteoarthritis or intervertebral disc disease can also include, for example, determining the level of RNA encoding the β-catenin fusion polypeptide, wherein a decrease in the level of expression of the RNA as compared to a control indirectly indicates a decrease in the level of the β-catenin fusion polypeptide, which indicates the agent reduces or prevents one or more symptoms of osteoarthritis or intervertebral disc disease.

The level of protein expression is determined using an assay selected from the group consisting of Western blot, enzyme-linked immunosorbent assay (ELISA), enzyme immunoassay (EIA), radioimmunoassay (RIA), or protein array. The level of RNA expression is determined using an assay selected from the group consisting of microarray analysis, gene chip, Northern blot, in situ hybridization, reverse transcription-polymerase chain reaction (RT-PCR), one step PCR, and quantitative real time (qRT)-PCR. The analytical techniques to determine protein or RNA expression are known. See, e.g. Sambrook et al., Molecular Cloning: A Laboratory Manual, 3^{rd} Ed., Cold Spring Harbor Press, Cold Spring Harbor, N.Y. (2001).

Optionally, determining whether the agent reduces or prevents one or more symptoms of osteoarthritis or intervertebral disc disease can, for example, include determining the activity of the β-catenin fusion polypeptide. A decrease in the activity of the β-catenin fusion polypeptide as compared to a control indicates the agent reduces or prevents one or more symptoms of osteoarthritis or intervertebral disc disease. A decrease in the activity of the β-catenin fusion polypeptide can, for example, be determined by detecting the level of expression of one or more β-catenin regulated genes (e.g., aggrecan, Mmp-9, Mmp-13, Alp, Oc, colX, Bmp2, Wnt11, Wnt5, WISP, sFRP2, Adamts4, Adamts5, col9, Wnt7a, Wnt1, and Wnt3a). A decrease in the expression of one or more of aggrecan, Mmp-9, Mmp-13, Alp, Oc, colX, Bmp2, Wnt11, Wnt5, WISP, sFRP2, Adamts4, and Adamts5 as compared to a control indicates a decrease in the activity of the β-catenin fusion polypeptide. An increase in the expression of one or more of col9, Wnt7a, Wnt1, and Wnt3a as compared to a control indicates a decrease in the activity of the β-catenin fusion polypeptide. The level of expression can be detected, for example, by determining the level of protein or RNA expression.

Also provided herein is a method of screening for an agent that reduces or prevents osteoarthritis or intervertebral disc disease comprising the steps of: (a) providing a transgenic animal whose genome comprises a first nucleic acid sequence comprising SEQ ID NO:1 and a second nucleic acid sequence comprising SEQ ID NO:3; (b) administering to the transgenic animal an agent to be tested; and (c) determining whether the agent reduces or prevents one or more symptoms of osteoarthritis or intervertebral disc disease. Such symptoms include pain (commonly in hands, hips, knees, spine, or feet), stiffness after periods of inactivity, limited joint motion, tenderness and occasional swelling, joint deformity, joint cracking, osteophyte formation, reduced cartilage or joint space, etc.

Also provided herein is a method of screening for an agent that reduces or prevents osteoarthritis or intervertebral disc disease comprising the steps of: (a) providing a cell comprising (i) a first nucleic acid sequence encoding a fusion polypeptide, wherein the fusion polypeptide comprises a Cre recombinase and a mutated ligand binding domain of human estrogen receptor (CreER), wherein the first nucleic acid is operably linked to a chondrocyte-specific promoter, and (ii) a second nucleic acid sequence comprising a β-catenin fusion polypeptide; (b) contacting the cell with an agent to be tested; and (c) determining the level of expression or activity of the β-catenin fusion polypeptide in the cell. A decrease in expression or activity of the β-catenin fusion polypeptide indicates the agent reduces or prevents one or more symptoms of osteoarthritis or intervertebral disc disease. Determining the level of expression can, for example, include determining the level of RNA or protein expression, as described previously. Optionally, the method of screening further comprises obtaining a cell from a transgenic animal whose genome comprises a first nucleic acid sequence encoding a fusion polypeptide, wherein the fusion polypeptide comprises a Cre recombinase and a mutated ligand binding domain of human estrogen receptor (CreER), wherein the first nucleic acid is operably linked to a chondrocyte-specific promoter, and a second nucleic acid sequence comprising a β-catenin fusion polypeptide. The cell obtained from the transgenic animal can, for example, be a chondrocyte or a fibroblast.

Also provided herein is a method of identifying a subject with or at risk for developing osteoarthritis or intervertebral disc disease comprising the steps of: (a) obtaining a biological sample from the subject; and (b) determining the level of expression or activity of β-catenin in the sample. An increase in β-catenin expression or activity as compared to a control indicates the subject has or is at risk for developing osteoarthritis or intervertebral disc disease. The biological sample can, for example, comprise chondrocytes or fibroblasts. Determining the level of expression of β-catenin can, for example, include determining the level of RNA or protein expression, as described previously.

Optionally, the method further comprises determining the level of expression or activity of one or more of aggrecan, Mmp-13, alkaline phosphatase (Alp), osteocalcin (Oc), type X collagen (colX), Bmp2, Wnt5, Wnt11, sFRP2, WISP1, Adamts4, or Adamts5. An increase in the level of expression or activity of one or more of aggrecan, Mmp-9, Mmp-13, Alp, Oc, colX, Bmp2, Wnt5, Wnt11, sFRP2, WISP1, Adamts4, or Adamts5 indicates the subject has or is at risk for developing osteoarthritis or intervertebral disc disease. Optionally, the method further comprises determining the level of expression or activity of one or more of col9, Wnt1, Wnt3a, or Wnt7a. A decrease in the expression or activity of one or more of col9, Wnt1, Wnt3a, or Wnt7a indicates the subject has or is at risk for developing osteoarthritis or intervertebral disc disease.

Provided herein is a method of treating or preventing osteoarthritis or intervertebral disc disease in a subject comprising: (a) selecting a subject with or at risk of developing osteoarthritis or intervertebral disc disease; and (b) administering to the subject an effective amount of a first therapeutic agent comprising a β-catenin inhibitor or a MMP-13 inhibitor. Optionally, the subject has osteoarthritis and the first therapeutic agent comprises a β-catenin inhibitor. Optionally, the subject has intervertebral disc disease and the first therapeutic agent comprises a MMP-13 inhibitor. The β-catenin inhibitor or MMP-13 inhibitor can, for example, be selected from the group consisting of a small molecule, a nucleic acid molecule, a polypeptide, a peptidomimetic, or a combination thereof. Optionally, the β-catenin inhibitor can be a small molecule. Optionally, the MMP-13 inhibitor can, for example, be a small molecule. Optionally, the small molecule comprises a Wnt3a antagonist or a Runx2 antagonist. Optionally, the β-catenin inhibitor or MMP-13 inhibitor can, for example, be a nucleic acid molecule. A nucleic acid molecule can, for example, be selected from the group consisting of a short interfering RNA (siRNA) molecule, a microRNA (miRNA) molecule, or an antisense molecule. Optionally, the β-catenin inhibitor or MMP-13 inhibitor can be a polypeptide. A polypeptide can, for example, be an antibody. A polypeptide can also, for example, be selected from the group consisting of secreted frizzled-related protein 3 (sFRP3) or glycogen synthase kinase-3β (GSK-3β).

As used herein, a β-catenin or MMP-13 inhibitory nucleic acid sequence can also be a short-interfering RNA (siRNA) sequence or a micro-RNA (miRNA) sequence. A 21-25 nucleotide siRNA or miRNA sequence can, for example, be produced from an expression vector by transcription of a short-hairpin RNA (shRNA) sequence, a 60-80 nucleotide precursor sequence, which is subsequently processed by the cellular RNAi machinery to produce either a siRNA or miRNA sequence. Alternatively, a 21-25 nucleotide siRNA or miRNA sequence can, for example, be synthesized chemically. Chemical synthesis of siRNA or miRNA seuquences is commercially available from such corporations as Dharmacon, Inc. (Lafayette, Colo.), Qiagen (Valencia, Calif.), and Ambion (Austin, Tex.). A siRNA sequence preferably binds a unique sequence within the β-catenin mRNA with exact complementarity and results in the degradation of the β-catenin mRNA molecule. A siRNA sequence can bind anywhere within the β-catenin mRNA molecule. Optionally, the β-catenin siRNA sequence can target the sequence 5′-AAGGCUUUUCCCAGUCCUUCA-3′ (SEQ ID NO:4), corresponding to nucleotides 203-223 of the mouse β-catenin mRNA nucleotide sequence, wherein position 1 begins with the first nucleotide of the coding sequence of the β-catenin mRNA molecule at Accession Number NM_{—}007614 at www.pubmed.gov. Optionally the β-catenin siRNA sequence can target the sequence 5′-AAGAUGAUGGUGUGCCAAGUG-3′ (SEQ ID NO:5) corresponding to nucleotides 1303-1323 of the mouse β-catenin mRNA nucleotide sequence. Optionally, the MMP-13 siRNA sequence can target the sequence 5′-CUGCGACUCUUGCGGGAAU-3′ (SEQ ID NO:6), corresponding to nucleotides 149-167 of the mouse MMP-13 mRNA nucleotide sequence, wherein position 1 begins with the first nucleotide of the coding sequence of the mRNA molecule at Accession Number NM_{—}008607 at www.pubmed.gov. Optionally, the MMP-13 siRNA sequence can target the sequence 5′-UCAAAUGGUCCCAAACGAA-3′ (SEQ ID NO:7), corresponding to nucleotides 335-353 of the mouse MMP-13 mRNA nucleotide sequence. Optionally, the MMP-13 siRNA sequence can target the sequence 5′-AGACUAUGGACAAAGAUUA-3′ (SEQ ID NO:8), corresponding to nucleotides 1232-1250 of the mouse MMP-13 mRNA nucleotide sequence. Optionally, the MMP-13 siRNA sequence can target the sequence 5′-GGCCCAUACAGUUUGAAUA-3′ (SEQ ID NO:9), corresponding to nucleotides 1340-1358 of the mouse MMP-13 mRNA nucleotide sequence. A miRNA sequence preferably binds a unique sequence within the β-catenin mRNA with exact or less than exact complementarity and results in the translational repression of the β-catenin mRNA molecule. A miRNA sequence can bind anywhere within the β-catenin mRNA sequence, but preferably binds within the 3′ untranslated region of the β-catenin mRNA molecule. Methods of delivering siRNA or miRNA molecules are known in the art. See, e.g., Oh and Park, Adv. Drug. Deliv. Rev. 61(10):850-62 (2009); Gondi and Rao, J. Cell Physiol. 220(2):285-91 (2009); and Whitehead et al., Nat. Rev. Drug. Discov. 8(2):129-38 (2009).

As used herein, a β-catenin inhibitory nucleic acid sequence can be an antisense nucleic acid sequence. Antisense nucleic acid sequences can, for example, be transcribed from an expression vector to produce an RNA which is complementary to at least a unique portion of the β-catenin mRNA and/or the endogenous gene which encodes β-catenin. Hybridization of an antisense nucleic acid under specific cellular conditions results in inhibition of β-catenin protein expression by inhibiting transcription and/or translation.

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boemer et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in

Immunol. 7:33 (1993)).

Provided herein are methods of treating or preventing osteoarthritis or intervertebral disc disease in a subject. Such methods include administering an effective amount of a β-catenin inhibitor or a MMP-13 inhibitor comprising a small molecule, a polypeptide, a nucleic acid molecule, a peptidomimetic or a combination thereof. Optionally, the small molecules, polypeptides, nucleic acid molecules, and/or peptidomimetics are contained within a pharmaceutical composition.

Provided herein are compositions containing the provided small molecules, polypeptides, nucleic acid molecules, and/or peptidomimetics and a pharmaceutically acceptable carrier described herein. The herein provided compositions are suitable of administration in vitro or in vivo. By pharmaceutically acceptable carrier is meant a material that is not biologically or otherwise undesirable, i.e., the material is administered to a subject without causing any undesirable biological effects or interacting in a deleterious manner with the other components of the pharmaceutical composition in which it is contained. The carrier is selected to minimize any degradation of the active ingredient and to minimize any adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. Optionally, the composition is administered by oral inhalation, nasal inhalation, or intranasal mucosal administration. Administration of the compositions by inhalant can be through the nose or mouth via delivery by spraying or droplet mechanism, for example, in the form of an aerosol.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retorviruses, Cold Spring Harbor Laboratory Press (1997), which is incorporated by reference herein for the vectors and methods of making them. The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

The method of treating or preventing osteoarthritis or intervertebral disc disease in a subject can further comprise administering one or more second therapeutic agents to the subject. The second therapeutic agent can, for example, be selected from the group consisting of pain relievers, non-steroidal anti-inflammatory drugs (NSAID), and corticosteroids. A pain reliever can, for example, be a narcotic selected from the group consisting of tramadol, hydrocodone, oxycodone, and morphine. Additionally, a pain reliever can be selected from the group consisting of paracetamol, acetaminophen, and capsaicin. A NSAID can, for example, be selected from the group consisting of diclofenac, ibuprofen, naproxen, ketoprofen, and celecoxib. Optionally, the second therapeutic can, for example, be selected from the group consisting of glucocorticoid, hyaluronan, glucosamine, chondroitin, omega-3 fatty acid, boswellia, bromelain, an antioxidant, hydrolyzed collagen, ginger extract, selenium, vitamin B9, vitamin B12, and BMP-6.

Any of the aforementioned second therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., osteoarthritis or intervertebral disc disease). The term patient or subject includes human and veterinary subjects.

A subject at risk of developing a disease or disorder can be genetically predisposed to the disease or disorder, e.g., have a family history or have a mutation in a gene that causes the disease or disorder, or show early signs or symptoms of the disease or disorder. A subject currently with a disease or disorder has one or more than one symptom of the disease or disorder and may have been diagnosed with the disease or disorder.

The methods and agents as described herein are useful for both prophylactic and therapeutic treatment. For prophylactic use, a therapeutically effective amount of the agents described herein are administered to a subject prior to onset (e.g., before obvious signs of osteoarthritis or intervertebral disc disease) or during early onset (e.g., upon initial signs and symptoms of osteoarthritis or intervertebral disc disease). Prophylactic administration can occur for several days to years prior to the manifestation of symptoms of osteoarthritis or intervertebral disc disease. Prophylactic administration can be used, for example, in the preventative treatment of subjects diagnosed with a genetic predisposition to osteoarthritis or intervertebral disc disease or after joint surgery or trauma. Therapeutic treatment involves administering to a subject a therapeutically effective amount of the agents described herein after diagnosis or development of osteoarthritis or intervertebral disc disease.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus , if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### General Methods

### Generation of Transgenic Mice

Col2a1-CreER^{T2} transgenic mice were bred with Rosa26 reporter mice. Methods for mouse genotyping including primer sequences are the same as described previously (Chen et al., Genesis 45:44-50 (2007); Zhu et al., Osteoarthr. Cartilage 16:129-30 (2008)). Tamoxifen (TM, 1 mg/10 g body weight/day, i.p. injection, ×5 days) was administered to the 3- and 6-month-old mice, which were sacrificed 2 months after TM induction at the age of 5 and 8 months. Cre-recombination efficiency was evaluated by X-Gal staining. Nuclear Fast Red staining was performed as a counter stain. β-catenin^{fx(Ex3)/fx(Ex3)} mice were originally reported by Harada et al (Harada et al., Embo J. 18:5931-42 (1999)). The sequences of PCR primers for genotyping β-catenin^{fx(Ex3)/fx(Ex3)} mice are: upper primer, 5′-AGGGTACCTGAAGCTCAGCG-3′ (SEQ ID NO:10) and lower primer, 5′-CAGTGGCTGACAGCAGCTTT-3′ (SEQ ID NO:11). The 412-bp PCR product was detected in wild-type mice, and the 645-bp PCR product was detected in homozygous β-catenin^{fx(Ex3)/fx(Ex3)} mice. In heterozygous mice (β-catenin^{fx(Ex3)/wt}), both 412 and 645-bp PCR products were detected. The Col2a1-CreER^{T2};β-catenin^{fx(Ex3)/w} transgenic mice and their Cre-negative littermates were used as controls and were administered TM as the experimental animals for phenotype analysis and cellular function studies.

### Histology and Histomorphometry

Initial X-ray and histological analyses were performed. Knee joints from 5 and 8-month-old Col2a1-CreER^{T2};β-catenin^{fx(Ex3)/wt} (β-catenin cAct) transgenic mice and Cre-negative control mice were dissected, fixed in 10% formalin, decalcified and embedded in paraffin. Serial mid-sagittal sections of knee joints were cut every 10 μm from both the medial and lateral compartments. The sections were stained with Alcian blue/Hemotoxylin & Orange G (AB/H&OG) and Safranin O/Fast green (SO/FG). To quantify changes in articular cartilage area and articular chondrocyte numbers, articular cartilage was outlined on the tibial surface and an area algorithm in the software ImagePro 4.5 (Leeds Precision Instruments; Minneapolis, Minn.) was used to determine the pixel area of outlined articular cartilage from each section. Using this approach, the average articular cartilage area was determined from 7 WT and β-catenin cAct knee joints.

Histologic changes in intervertebral disc tissues were evaluated by Safranin O/Fast green and Alcian blue/Hematoxylin & orange G staining in 1- and 3-month old Col2a1-CreER^{T2};β-Catenin^{fx(Ex3)/wt} and Col2a1-CreER^{T2};β-catenin^{fx(Ex3)/wt};Mmp13^{fx/fx} mice and compared with same aged β-catenin cACt mice and Mmp13 cKO mice. TM induction was performed when mice are at 2 weeks of age. Disc tissue endplate cartilage area of 1- and 3-month-old mice was analyzed.

### Immunostaining

Tissue sections were deparaffinized by immersing in xylene, then fixed with 4% paraformaldehyde for 15 minutes and treated with 0.5% Triton for 15 minutes followed by fixing with 4% paraformaldehyde for another 5 minutes. Sections were then incubated with a rabbit anti-β-catenin polyclonal antibody (1:20 dilution, Cell Signaling; Danvers, Mass.), goat anti-MMP-13 polyclonal antibody (1:100 dilution, Chemicon International; Temecula, Calif.) overnight and then a HRP-conjugated secondary antibody for 30 minutes. Slides were mounted with Permount (Electron Microscopy Sciences; Hatfield, Pa.) and visualized under a light microscope.

### Cell Isolation and Cell Culture

TM (1 mg/10 g body weight/day, i.p. injection, ×5 days) was administered into 1-month-old Col2a1-CreER^{T2};β-catenin^{fx(Ex3)/wt} transgenic mice and their Cre-negative littermates which were sacrificed 1 month after TM induction (2 months old). The mice were sacrificed and genotyped using tail tissues obtained at sacrifice.

The femoral articular cartilage caps were harvested, washed with PBS, and then digested with 0.1% Pronase (Roche Applied Science; Indianapolis, Ind.) in PBS and incubated for 30 minutes in a 37° C. shaking water bath. This was followed by incubation in a solution of 0.1% collagenase A (Roche Applied Science; Indianapolis, Ind.) in serum-free Dulbecco's modified Eagle's medium (DMEM) for 4 hours in a shaking water bath. The digestion solution was passed through 70 μm Swinnex filters to remove all residual fragments. The solution was centrifuged, and the cells were resuspended in complete medium (DMEM with 10% fetal bovine serum (FBS) and 1% penicillin/streptomycin). The media was changed every 3 days.

### Micro-CT Analysis

Changes in loss of endplate cartilage, osteophyte formation and disc space narrowing in β-catenin cAct mice were detected by micro-CT analysis. Formalin-fixed spine tissues from 1- and 3-month-old Col2a1-CreER^{T2};β-catenin^{fx(Ex3)/wt} mice and Col2a1-CreER^{T2};β-catenin^{fx(Ex3)/wt};Mmp13^{fx/fx} mice and same aged β-catenin cAct mice and Mmp13 cKO mice was evaluated by micro-CT using a SCANCO viva-CT40 scanner (SCANCO USA, Inc.; Southeastern, Pa.). Spine samples were scanned at a resolution of 12-μm with a slice increment of 10-μm. Images from each group were reconstructed at identical thresholds to allow 3-dimensional structural rendering of each spine sample. Morphometric analyses were performed on selected cervical and lumbar spine regions.

### Total RNA Extraction and Real-Time Reverse Transcription-Polymrease Chain Reaction (RT-PCR) Analysis

Total RNA extracted from primary articular chondrocytes, articular cartilage tissue, or primary disc cells was prepared using Trizol (Invitrogen; Carlsbad, Calif.) according to the manufacturer's protocol. One microgram total RNA was used to synthesize cDNA by iScripts cDNA Synthesis Kit (Bio-Rad; Hercules, Calif.). Primer names and sequences for real-time PCR are listed in Tables 1-3.

TABLE 1
Primer sequences for marker genes
of articular chondrocytes.

| | |
|---|---|
| Mmp-9 Fw | 5′-TGAATCAGCTGGCTTTTGTG-3′ |
| | (SEQ ID NO: 12) |
| Mmp-9 Rev | 5′-ACCTTCCAGTAGGGGCAACT-3′ |
| | (SEQ ID NO: 13) |
| Mmp-13 Fw | 5′-TTTGAGAACACGGGGAAGA-3′ |
| | (SEQ ID NO: 14) |
| Mmp-13 Rev | 5′-ACTTTGTTGCCAATTCCAGG-3′ |
| | (SEQ ID NO: 15) |
| Aggrecan Fw | 5′-AGGACCTGGTAGTGCGAGTG-3′ |
| | (SEQ ID NO: 16) |
| Aggrecan Rev | 5′-GCGTGTGGCGAAGAA-3′ |
| | (SEQ ID NO: 17) |
| Bmp2 Fw | 5′-GCTTTTCTCGTTTGTGGAGC-3′ |
| | (SEQ ID NO: 18) |
| Bmp2 Rev | 5′-TGGAAGTGGCCCATTTAGAG-3′ |
| | (SEQ ID NO: 19) |
| Bmp4 Fw | 5′-GAGGAGGAGGAAGAGCAGAG-3′ |
| | (SEQ ID NO: 20) |
| Bmp4 Rev | 5′-TGGGATGTTCTCCAGATGTT-3′ |
| | (SEQ ID NO: 21) |
| Bmp6 Fw | 5′-CTCAGAAGAAGGTTGGCTGG-3′ |
| | (SEQ ID NO: 22) |
| Bmp6 Rev | 5′-ACCTCGCTCACCTTGAAGAA-3′ |
| | (SEQ ID NO: 23) |
| Gdf5 Fw | 5′-TCCTTCCTGCTGAAGAAGACCA-3′ |
| | (SEQ ID NO: 24) |
| Gdf5 Rev | 5′-TAAAGCTGGTGATGGTGTTGGC-3′ |
| | (SEQ ID NO: 25) |
| Col1a1 Fw | 5′-GCATGGCCAAGAAGACATCC-3′ |
| | (SEQ ID NO: 26) |
| Col1a1 Rev | 5′-CCTCGGGTTTCCACGTCTC-3′ |
| | (SEQ ID NO: 27) |
| Col2a1 Fw | 5′-CCACACCAAATTCCTGTTCA-3′ |
| | (SEQ ID NO: 28) |
| Col2a1 Rev | 5′-ACTGGTAAGTGGGGCAAGAC-3′ |
| | (SEQ ID NO: 29) |
| ColXa1 Fw | 5′-ACCCCAAGGACCTAAAGGAA-3′ |
| | (SEQ ID NO: 30) |
| ColXa1 Rev | 5′-CCCCAGGATACCCTGTTTTT-3′ |
| | (SEQ ID NO: 31) |
| Osteocalcin Fw | 5′-AGGGAGGATCAAGTCCCG-3′ |
| | (SEQ ID NO: 32) |
| Osteocalcin Rev | 5′-GAACAGACTCCGGCGCTA-3′ |
| | (SEQ ID NO: 33) |
| Alp Fw | 5′-TCCTGACCAAAAACCTCAAAGG-3′ |
| | (SEQ ID NO: 34) |
| Alp Rev | 5′-TCGTTCATGCAGAGCCTGC-3′ |
| | (SEQ ID NO: 35) |
| Vegf Fw | 5′-CCTTGCTGCTCAACCTCCAC-3′ |
| | (SEQ ID NO: 36) |
| Vegf Rev | 5′-CACACAGGATGGCTTGAAGA-3′ |
| | (SEQ ID NO: 37) |

TABLE 2
Primer sequences for Wnt signaling genes.

| | |
|---|---|
| Wnt1 Fw | 5′-ACAGCGTTCATCTTCGCAATCACC-3′ |
| | (SEQ ID NO: 38) |
| Wnt1 Rev | 5′-AAATCGATGTTGTCACTGCAGCCC-3′ |
| | (SEQ ID NO: 39) |
| Wnt3a Fw | 5′-GGCTCCTCTCGGATACCTCT-3′ |
| | (SEQ ID NO: 40) |
| Wnt3a Rev | 5′-GGGCATGATCTCCACGTAGT-3′ |
| | (SEQ ID NO: 41) |
| Wnt4 Fw | 5′-CTCAAAGGCCTGATCCAGAG-3′ |
| | (SEQ ID NO: 42) |
| Wnt4 Rev | 5′-GTCCCTTGTGTCACCACCTT-3′ |
| | (SEQ ID NO: 43) |
| Wnt5 Fw | 5′-TGCATGATCCCATGCCCTTT-3′ |
| | (SEQ ID NO: 44) |
| Wnt5 Rev | 5′-ACCAAACAGCTGCAACACCT-3′ |
| | (SEQ ID NO: 45) |
| Wnt7a Fw | 5′-TACGTGCAAGTGAATGCGGT-3′ |
| | (SEQ ID NO: 46) |
| Wnt7a Rev | 5′-TGGTTCTTTCCCTGTGAGCA-3′ |
| | (SEQ ID NO: 47) |
| Wnt7b Fw | 5′-TTCCTCCACAACACATGGCA-3′ |
| | (SEQ ID NO: 48) |
| Wnt7b Rev | 5′-ATGCAAGGCAAGGGCAAACA-3′ |
| | (SEQ ID NO: 49) |
| Wnt11 Fw | 5′-TGCTATGGCATCAAGTGGCT-3′ |
| | (SEQ ID NO: 50) |
| Wnt11 Rev | 5′-CCAGCTGTTTACAGTGTTGCGT-3′ |
| | (SEQ ID NO: 51) |
| sFRP2 Fw | 5′-ATCCGCAAGCTGCAATGCTA-3′ |
| | (SEQ ID NO: 52) |
| sFRP2 Rev | 5′-TGTGCTTGGGAAACCGGAAA-3′ |
| | (SEQ ID NO: 53) |
| WISP1 Fw | 5′-TGGCCTGGTTCAAGGAAAGT-3′ |
| | (SEQ ID NO: 54) |
| WISP1 Rev | 5′-TGCCTTTGAGCTTCAGCGTT-3′ |
| | (SEQ ID NO: 55) |

TABLE 3
Primer sequences for primary disc
gene expression analysis.

| | |
|---|---|
| Col2a1 Fw | 5′-CCACACCAAATTCCTGTTCA-3′ |
| | (SEQ ID NO: 28) |
| Col2a1 Rev | 5′-ACTGGTAAGTGGGGCAAGAC-3′ |
| | (SEQ ID NO: 29) |
| Col9 Fw | 5′-TGGAAAGAACAAGCGCCACT-3′ |
| | (SEQ ID NO: 56) |
| Col9 Rev | 5′-TGCAAAGCCATCCGCATCAA-3′ |
| | (SEQ ID NO: 57) |
| ColXa1 Fw | 5′-ACCCCAAGGACCTAAAGGAA-3′ |
| | (SEQ ID NO: 30) |
| ColXa1 Rev | 5′-CCCCAGGATACCCTGTTTTT-3′ |
| | (SEQ ID NO: 31) |
| Alp Fw | 5′-TCCTGACCAAAAACCTCAAAGG-3′ |
| | (SEQ ID NO: 34) |
| Alp Rev | 5′-TCGTTCATGCAGAGCCTGC-3′ |
| | (SEQ ID NO: 35) |
| Aggrecan Fw | 5′-AGGACCTGGTAGTGCGAGTG-3′ |
| | (SEQ ID NO: 16) |
| Aggrecan Rev | 5′-GCGTGTGGCGAAGAA-3′ |
| | (SEQ ID NO: 17) |
| Mmp-2 Fw | 5′-TGGTCCGCGTAAAGTATGGGAA-3′ |
| | (SEQ ID NO: 58) |
| Mmp-2 Rev | 5′-CTGCATTGCCACCCATGGTAAA-3′ |
| | (SEQ ID NO: 59) |
| Mmp-3 Fw | 5′-TCAGTGGATCTTCGCAGTTGGA-3′ |
| | (SEQ ID NO: 60) |
| Mmp-3 Rev | 5′-ACAGGATGCCTTCCTTGGATCT-3′ |
| | (SEQ ID NO: 61) |
| Mmp-13 Fw | 5′-TTTGAGAACACGGGGAAGA-3′ |
| | (SEQ ID NO: 14) |
| Mmp-13 Rev | 5′-ACTTTGTTGCCAATTCCAGG-3′ |
| | (SEQ ID NO: 15) |
| Adamts4 Fw | 5′-TCTGGCTTTAACGAGGAGCCTT-3′ |
| | (SEQ ID NO: 62) |
| Adamts4 Rev | 5′-GGCAAGCAGGGTTGGAATCTTT-3′ |
| | (SEQ ID NO: 63) |
| Adamts5 Fw | 5′-TGCATGGAGGCCATCATCTT-3′ |
| | (SEQ ID NO: 64) |
| Adamts5 Rev | 5′-TGCAAATGGCAGCACCAACA-3′ |
| | (SEQ ID NO: 65) |

### Human Tissue Procurement and Fixation

For human tissue, normal cartilage was collected from trauma/amputation patients and arthritic cartilage was collected from patients undergoing total knee arthroplasty. All human samples were harvested without patient identifiers.

Following recovery, human tissue was fixed for between 2 and 10 days in room temperature in 10% neutral-buffered formalin. All samples were decalcified in a solution containing 10% w/v EDTA for 3 weeks and embedded in paraffin. Embedded samples were cut with a microtome to generate 3 μm thick sections which were mounted on positively-charged slides, baked at 60° C. for 30 minutes, de-paraffinized in xylene and re-hydrated in decreasing concentrations of ethanol.

### Mankin Scoring in Human Tissue

Human tissue sections were stained with Safranin O/fast green and graded using a modified version of the Mankin scale (Mankin et al., J. Bone Joint Surg. Am. 53:523-37 (1971)). Specifically, cartilage was assigned a grade as follows: 0=normal cartilage; 1=localized fibrillation; 2=broadly distributed fibrillation; 3=clefts to the transitional zone; 4=clefts to radial zone; 5=clefts to calcified cartilage; and 6=complete disorganization. Two independent observers assigned grades to all samples studied and the distribution of averaged grades allowed for stratification of arthritic samples into 2 groups: low Mankin grade (mild/early osteoarthritis (OA), grade 1.7) and high Marlin grade (severe OA, grade 5.0). Expression of β-catenin protein was examined by immunohistochemical method.

### Example 1

### Tamoxifen (TM)-Induced Cre-Recombination was Achieved in Adult Col2a1-CreER^{T2} Transgenic Mice

In previous studies, efficient Cre-recombination in articular chondrocytes after

TM induction at early postnatal stages (TM was administered in the 2-week-old mice) was demonstrated (Zhu et al., Osteoarthr. Cartilage 16:129-30 (2008)). In the present study, TM-induced Cre-recombination in fully developed growth-plate cartilage was investigated. Thus, creating the possibility of completely separating the role of a specific gene in articular chondrocytes from its potential effect on the growth plate cartilage, which may indirectly affect the function of articular cartilage. Col2a1-CreER^{T2} transgenic mice were bred with Rosa26 reporter mice (Soriano, Nat. Genet. 21:70-71 (1999); Mao et al., Proc. Natl. Acad. Sci. USA 96:5037-42 (1999)). TM induction was performed in the 3- and 6-month-old Col2a1-CreER^{T2};R26R mice. Mice were then sacrificed 2 months after TM induction at the age of 5 and 8 months and Cre-recombination efficiency was evaluated by X-Gal staining.

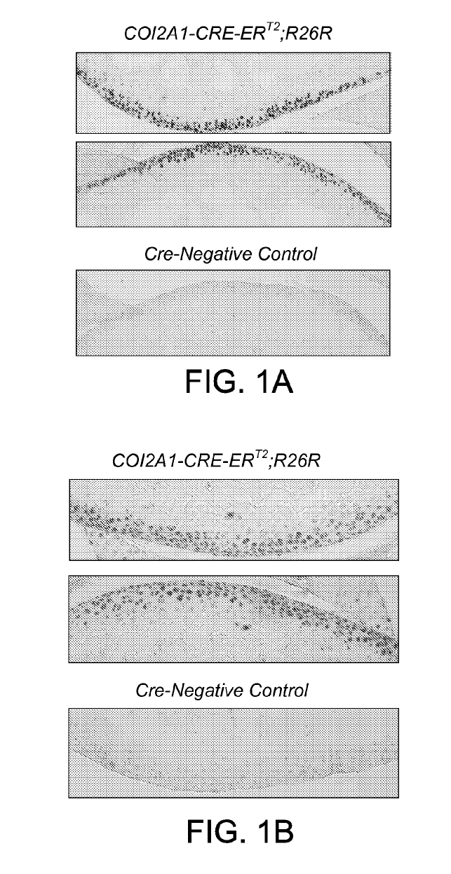

Growth plate cartilage is fully developed when mice have reached 3 months of age. When TM was administered in the 3-month-old mice, an average of 84% (n=3) Cre-recombination efficiency was achieved in articular chondrocytes 2 months after TM induction as determined by X-Gal staining (FIG. 1A). Similar but slightly lower recombination efficiency (76%) (n=3) was achieved in articular chondrocytes when TM was administered in the 6-month-old Col2a1-CreER^{T2};R26R mice followed by X-Gal staining 2 months later (FIG. 1B). In contrast, less than 20% Cre-recombination efficiency was observed in growth plate chondrocytes in these mice.

### Example 2

### OA-Like Articular Cartilage Destruction was Observed in β-Catenin cAct Mice

Col2a1-CreER^{T2} transgenic mice were bred with β-catenin^{fx(Ex3)/fx(Ex3)} mice to generate Col2a1-CreER^{T2};β-catenin^{fx(Ex3)/wt} (β-catenin cAct) mice. Since amino acids encoded by exon 3 contain critical GSK-3β phosphorylation sites, deletion of exon 3 of the β-catenin gene results in the production of a stabilized fusion protein which is resistant to phosphorylation by GSK-3β (Harada et al., EMBO J. 18:5931-42 (1999)). Three- and 6-month-old β-catenin cAct mice and Cre-negative control mice were treated with TM. The mice were sacrificed 2 months after TM induction and the increase of β-catenin protein levels in articular chondrocytes was detected in the 5-month-old β-catenin cAct mice compared to their Cre-negative controls (FIG. 2).

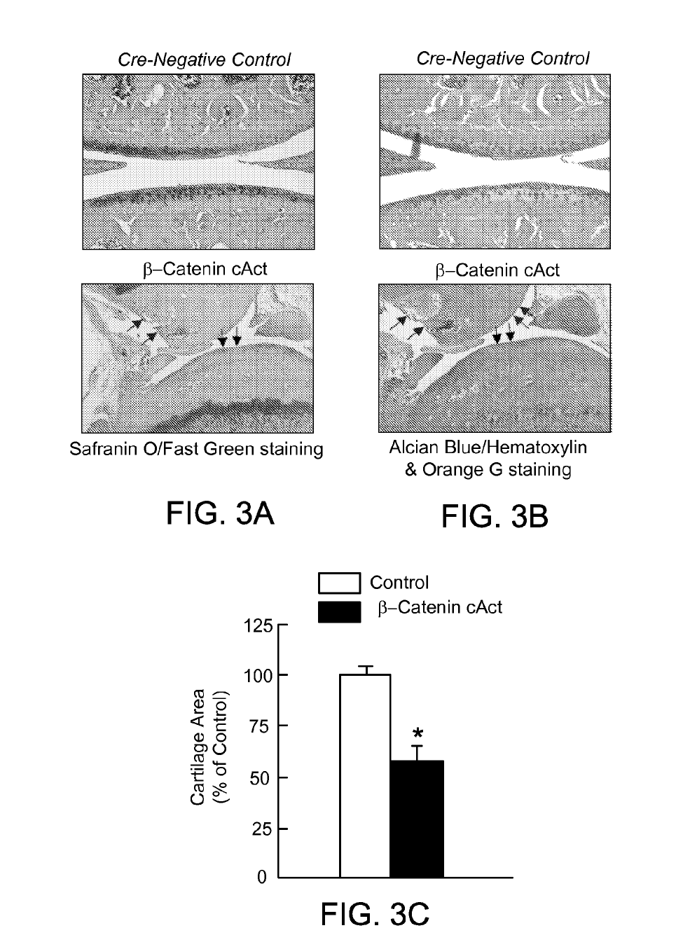

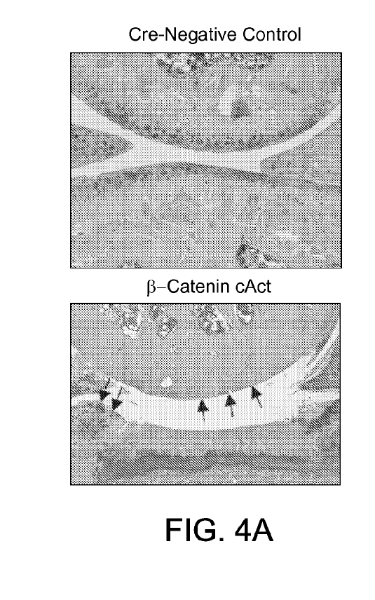

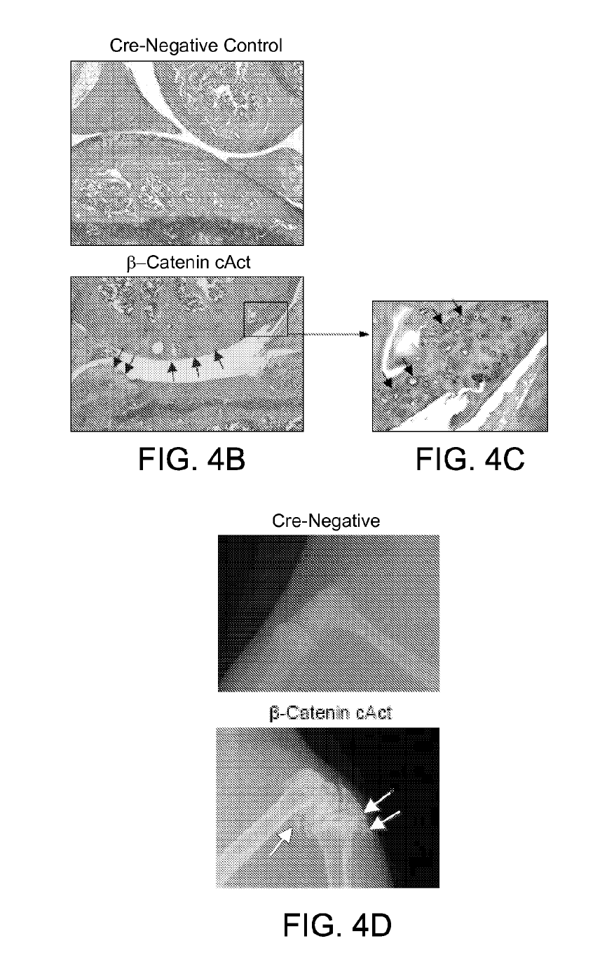

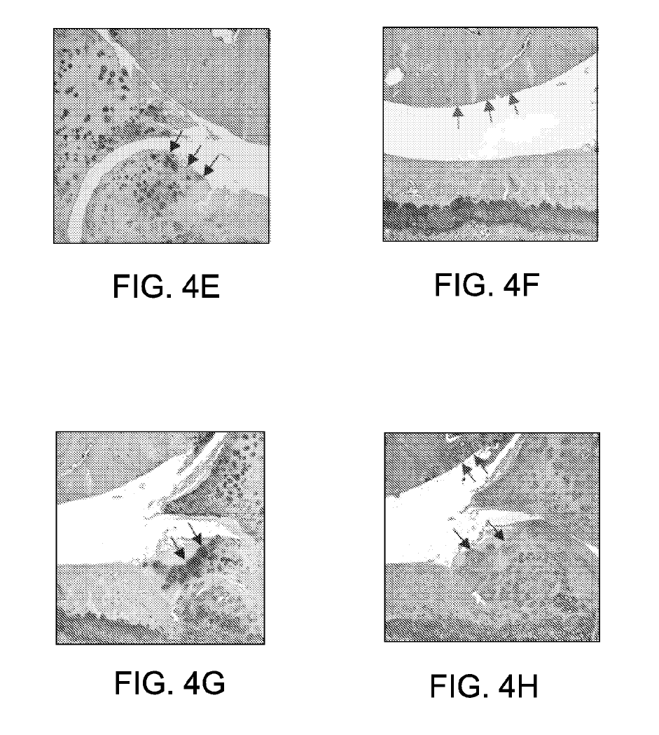

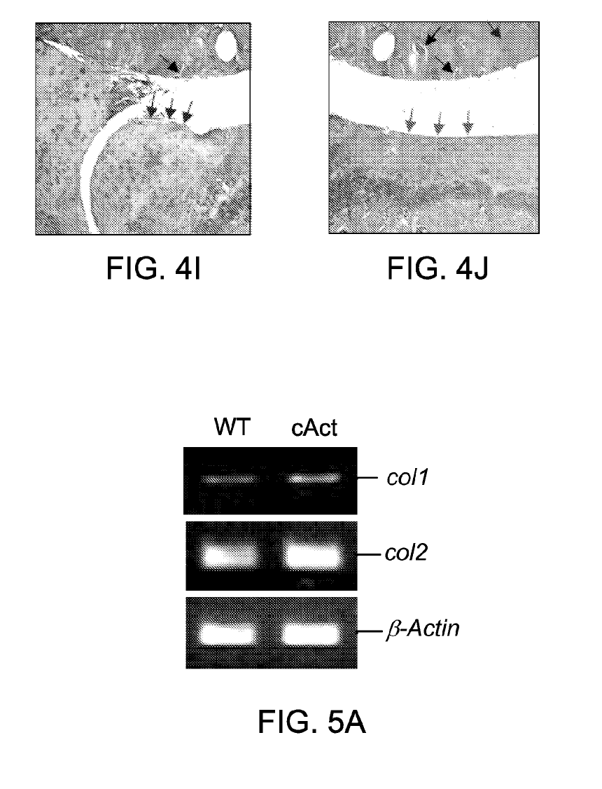

The articular cartilage phenotype of β-catenin cAct mice was analyzed by histology. Safranin 0/Fast green and Alcian blue/Hematoxylin & orange G staining was performed on 3 μm thick formalin-fixed sections. Histological results showed that age-dependent progressive loss of the smooth surface of articular cartilage occurs in β-catenin cAct mice. At the age of 5 months, mild degeneration was observed at the articular surface of knee joints. The Safranin O and Alcian blue staining was reduced and articular chondrocytes were missing in the weight-bearing area of the articular surface in β-catenin cAct mice (FIGS. 3A and 3B). Histomorphometric analysis showed that articular cartilage area was reduced in β-catenin cAct mice (FIG. 3C). At 8 months of age, destruction of articular cartilage was observed in β-catenin cAct mice. Cell cloning, surface fibrillation and vertical clefts, and formation of chondrophytes and osteophytes were observed (FIGS. 4A-4J). Complete loss of articular cartilage layers and the formation of new woven bone in response to the loss of subchondral bone were also found in β-catenin cAct mice (FIGS. 4A-4J). Histological analysis showed that 8 out of 8 (100%) and 7 out of 8 (87%) of the 5- and 8-month-old β-catenin cAct mice have articular cartilage destruction. In contrast, no articular cartilage damage was found in 5-month-old Cre-negative control mice and only minor articular cartilage damage was found in 1 out of 8 of 8-month-old Cre-negative control mice. Overall, these phenotypic changes resemble the clinical features commonly observed in OA patients.

### Example 3

### Articular Chondrocyte Maturation is Accelerated in β-Catenin cAct Mice

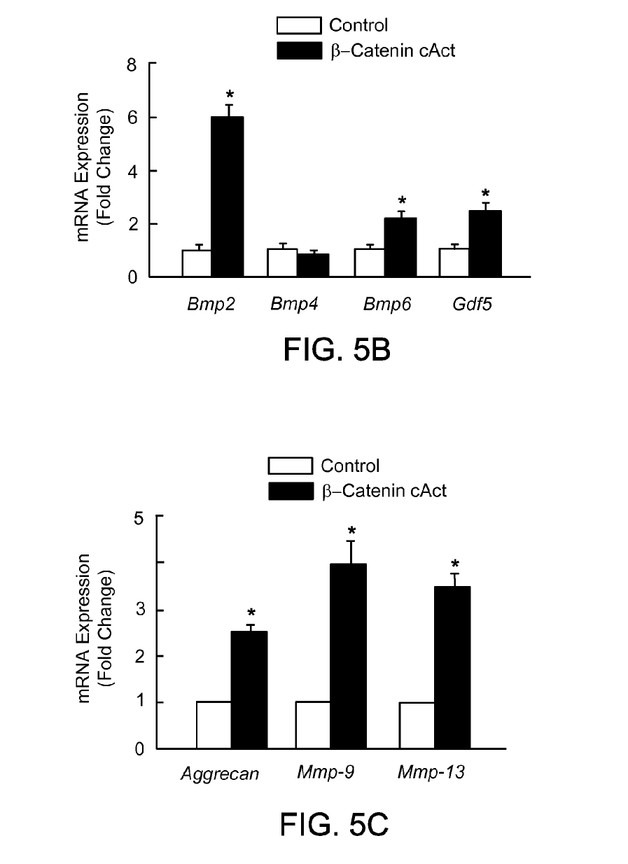

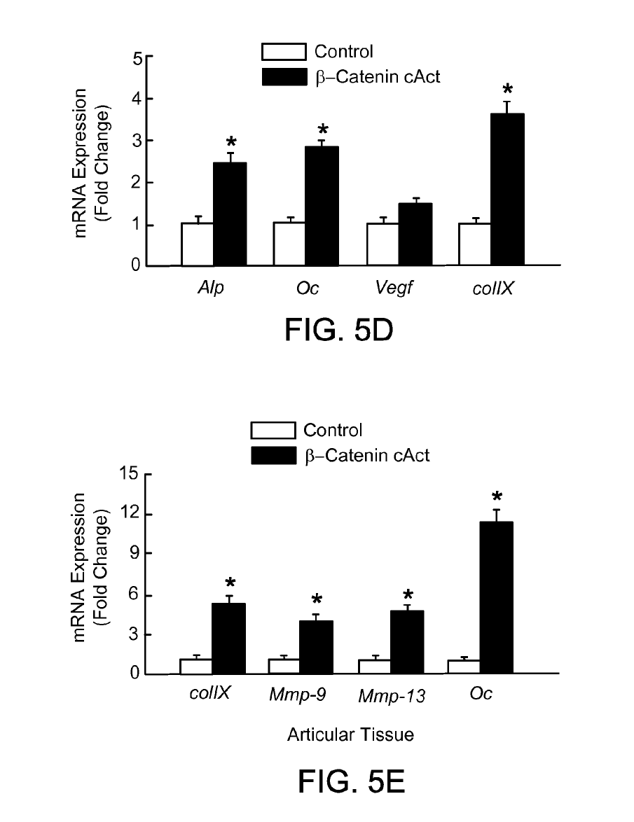

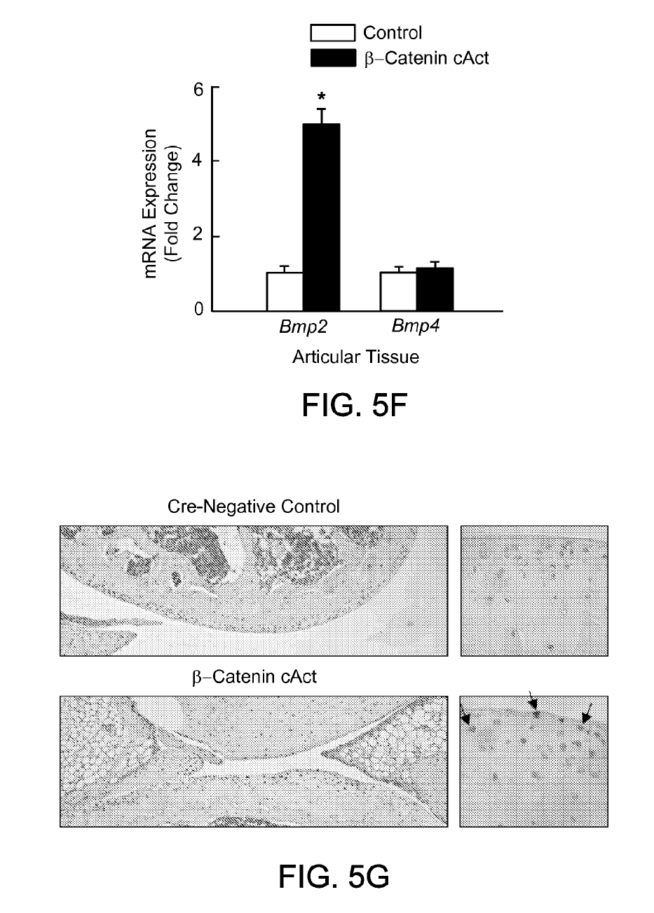

To determine changes in the maturation status of articular chondrocytes in β-catenin cAct mice, primary articular chondrocytes were isolated from 2-month-old β-catenin cAct mice and Cre-negative control mice in which TM induction was performed at the age of 1 month. Rounded cell morphology and expression of very low levels of type I collagen (col1) indicated that there was minimal fibroblast or osteoblast contamination of the primary articular chondrocyte cultures (FIG. 5A). The expression of articular chondrocyte marker genes was analyzed by quantitative real-time PCR (qRT-PCR). The expression of Bmp family members was first examined. Among them, Bmp2 was significantly up regulated (6-fold increase) (FIG. 5B). There was a greater than 2-fold increases in the expression of Bmp6 and Gdf5 (FIG. 5B). In contrast, Bmp4 expression was not changed (FIG. 5B). The expression of aggrecan was also increased 2.5-fold (FIG. 5C). The expression of two important matrix metalloproteases, Mmp-9 and Mmp-13, was also significantly increased (4 and 3.5-fold, respectively) (FIG. 5C). The mRNA levels of other chondrocyte maturation markers, such as alkaline phosphatase (Alp) (2.5-fold), osteocalcin (Oc, 3-fold) and type X collagen (colX, 3.5-fold) were also significantly increased (FIG. 5D). To further confirm if articular chondrocyte maturation is accelerated in β-catenin cAct mice, articular tissues from the 1-month-old β-catenin cAct mice and Cre-negative control mice were isolated. Total RNA was extracted from these tissues and the expression of chondrocyte marker genes was examined by real-time PCR. The results showed that the expression of colX β-fold), Mmp-9 (2-fold), Mmp-13 (3-fold) and Oc (12-fold) was significantly increased in β-catenin cAct mice (FIG. 5E). Consistent with gene expression from isolated articular chondrocytes, the expression of Bmp2, but not Bmp4, was significantly increased (5-fold) in articular tissues derived from β-catenin cAct mice (FIG. 5F). Immunostaining of sections from 8-month-old β-catenin cAct and Cre-negative controls demonstrated that MMP-13 protein levels are significantly increased in β-catenin cAct mice (FIG. 5G). Taken together, these findings clearly indicate that the chondrocyte maturation process is accelerated in β-catenin cAct mice.

### Example 4

### Alterations in Expression of Wnt Ligands and Wnt Antagonists

To determine if conditional activation of the β-catenin gene causes changes in

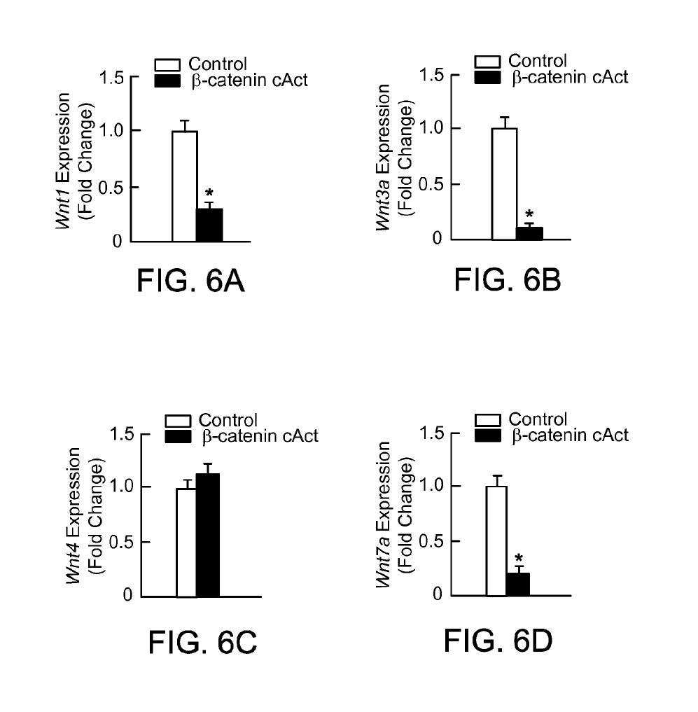

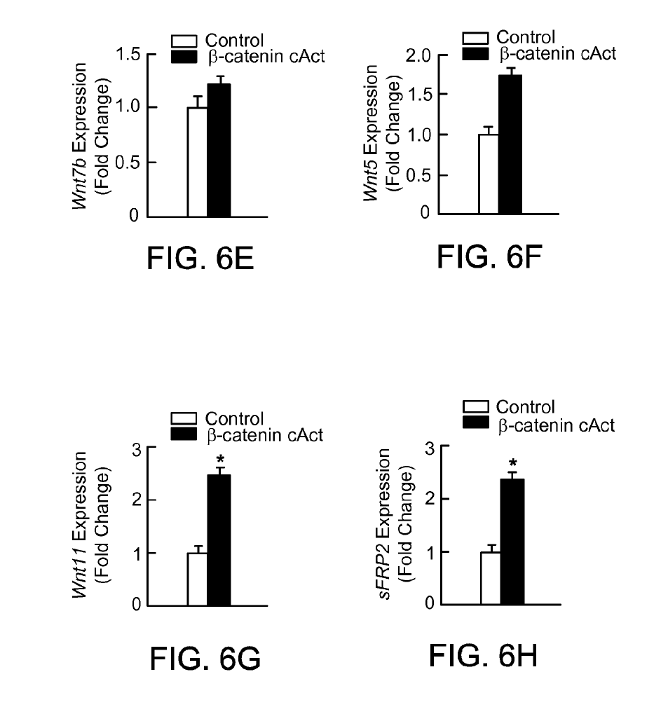

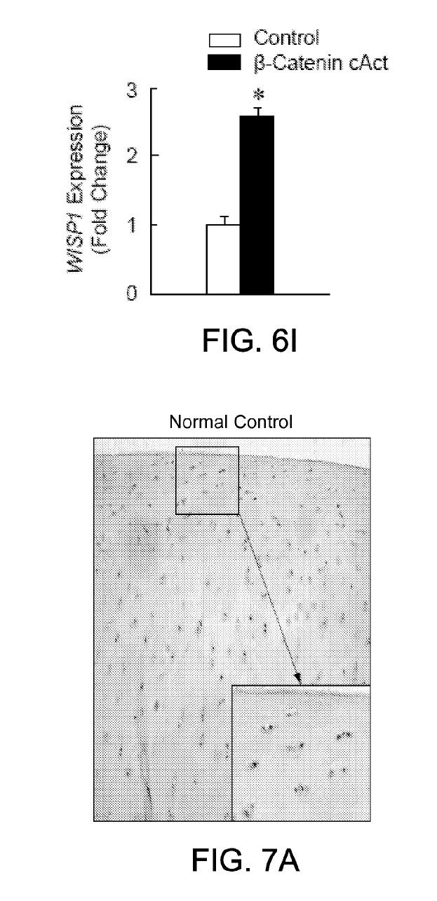

Wnt signaling, changes in expression of Wnt ligands and antagonists, which are involved in canonical and non-canonical Wnt signaling in articular chondrocytes, were analyzed. Primary articular chondrocytes were isolated from 1-month-old β-catenin cAct mice and Cre-negative control mice in which TM induction was performed at the age of 2 weeks. The expression of Wnt1, Wnt3a, and Wnt7a was significantly reduced (FIGS. 6A, 6B, and 6D), while no significant changes were found in the expression of Wnt4 and Wnt7b (FIGS. 6C and 6E) in articular chondrocytes derived from β-catenin cAct mice. In contrast, expression of Wnt5 and Wnt11 was significantly increased in articular chondrocytes in which β-catenin signaling is activated (FIGS. 6F and 6G). In contrast to the Wnt ligands, expression of the Wnt antagonist sFRP2 and the Wnt target gene WISP1 was also significantly increased in articular chondrocytes derived from β-catenin cAct mice (FIGS. 6H and 6I).

### Example 5

### β-Catenin Levels are Increased in Human OA Samples

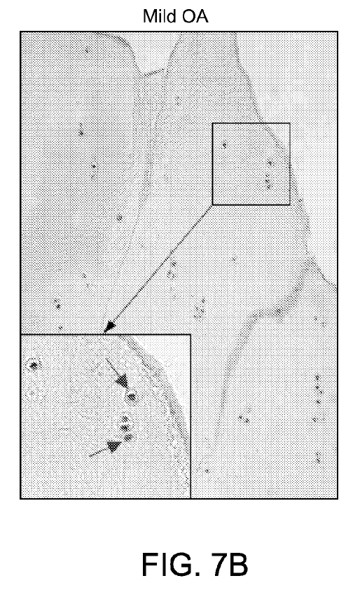

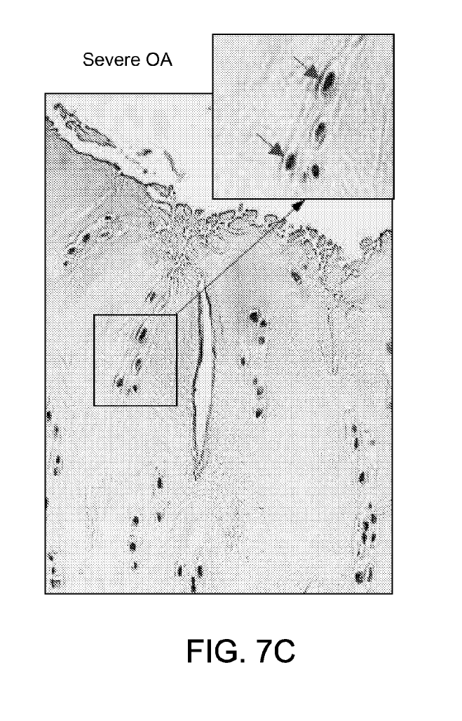

Using immunostaining methods, activation of β-catenin signaling in human OA samples was determined. Articular cartilage samples from patients undergoing total knee arthroplasty (OA samples) and from trauma/amputation patients (negative controls) were processed for Mankin grading to determine severity of osteoarthritis (Mankin et al., J. Bone Joint Surg. Am. 53:523-37 (1971)) and immunohistochemical analysis with an anti-β-catenin monoclonal antibody. The initial Mankin grading facilitated the stratification of OA samples into two groups: low Mankin grade (mild/early OA, average grade of 1.7, range 0-2.7) and high Mankin grade (severe OA, average grade of 5.0, range 3.3-8.7). While the normal cartilage group showed no significant immunoreactivity with the β-catenin antibody (FIG. 7A), both the low and high Mankin-graded OA groups displayed a significant cellular β-catenin staining (FIGS. 7B and 7C). Immunograding of all samples revealed a significant up-regulation of β-catenin in both the low and high Mankin groups compared to the normal control. These results establish a strong association between human OA and β-catenin expression.

### Example 6

### Tamoxifen (TM)-induced Cre-Recombination was Achieved in Postnatal and Adult Col2a1-CreER^{T2} Transgenic Mice

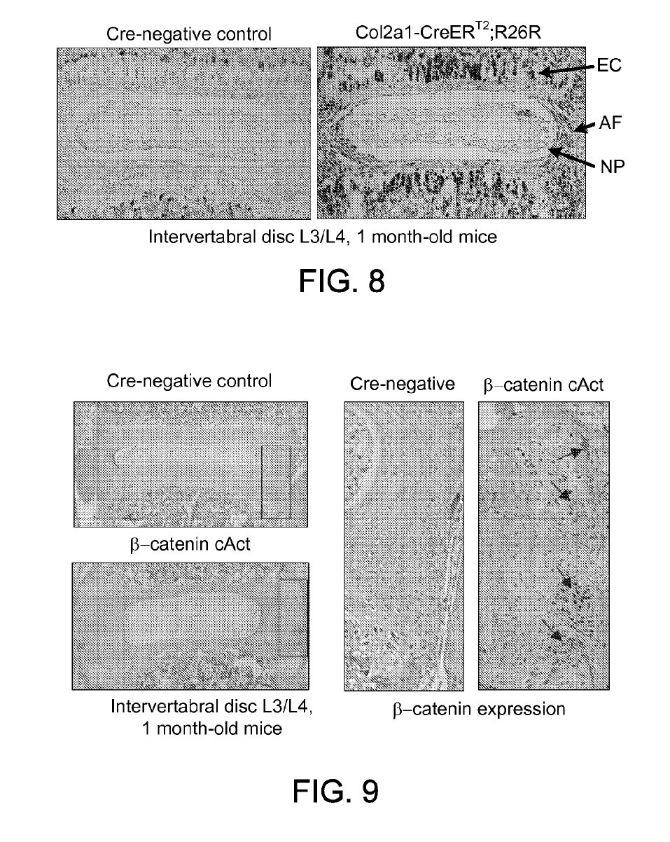

Since chondrocyte-specific β-catenin cAct mice (targeted by Col2a1-Cre transgenic mice) are embryonic lethal, to target intervertebral disc (IVD) cells (Col2a1-positive cell population) in postnatal and adult mice, the Col2a1-CreER^{T2};R26R transgenic mice were used. Cre-recombination efficiency in IVD cells was determined in postnatal mice. TM (1 mg/10 g body weight, i.p., ×5 days) was administered into 2-week-old Col2a1-CreER^{T2};R26R transgenic mice. Mice were sacrificed at 1 month of age and X-Gal staining was performed. The results showed high efficiency of Cre-recombination in annulus fibrosus cells and endplate cartilage cells but not in nucleus pulposus cells (maybe due to poor TM penetration into the nucleus pulposus area) (FIG. 8).

### Example 7

### Loss of Endplate Cartilage Tissue Destruction was Observed in β-Catenin cAct Mice

To study the effect of β-catenin activation on IVD cells, the Col2a1-CreER^{T2};β-catenin^{fx(Ex3)/wt} mice were used. The mice (2-week-old) were treated with TM (1 mg/10 g body weight, i.p.) for 5 days. Mice were sacrificed at 1 month of age and β-catenin immunostaining was performed. β-catenin over-expression was detected in disc cells of Col2a1-CreER^{T2};β-catenin^{fx(Ex3)/wt} mice which received TM treatment (β-catenin cAct mice). Due to the loss of endplate cartilage cells in 1-month-old β-catenin cAct mice, β-catenin over-expression was mainly detected in annulus fibrosus cells (FIG. 9).

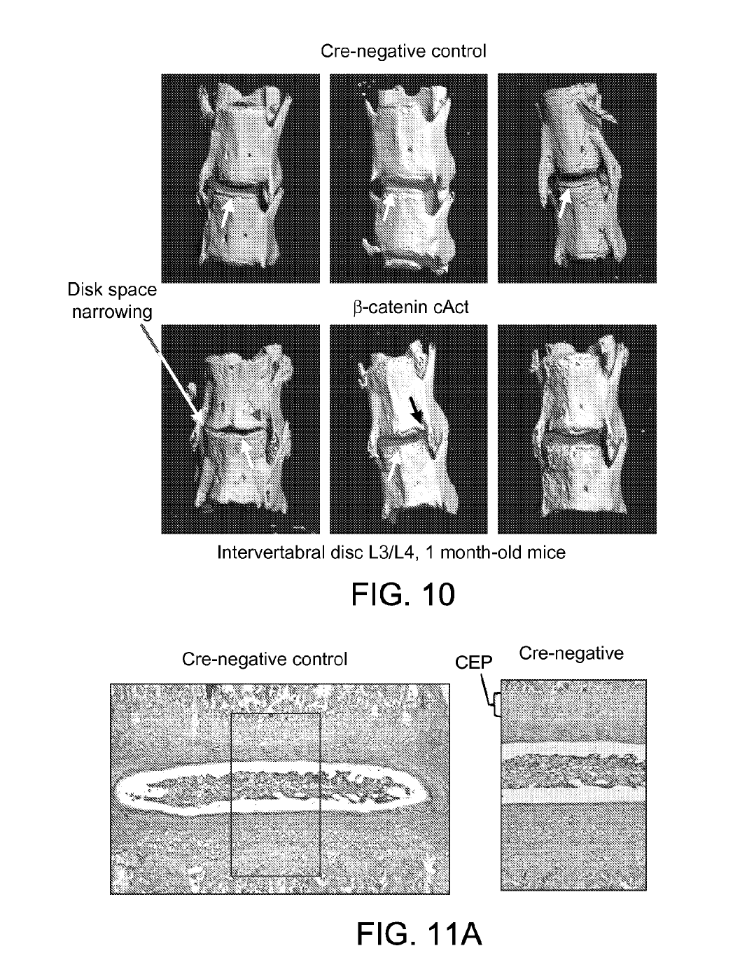

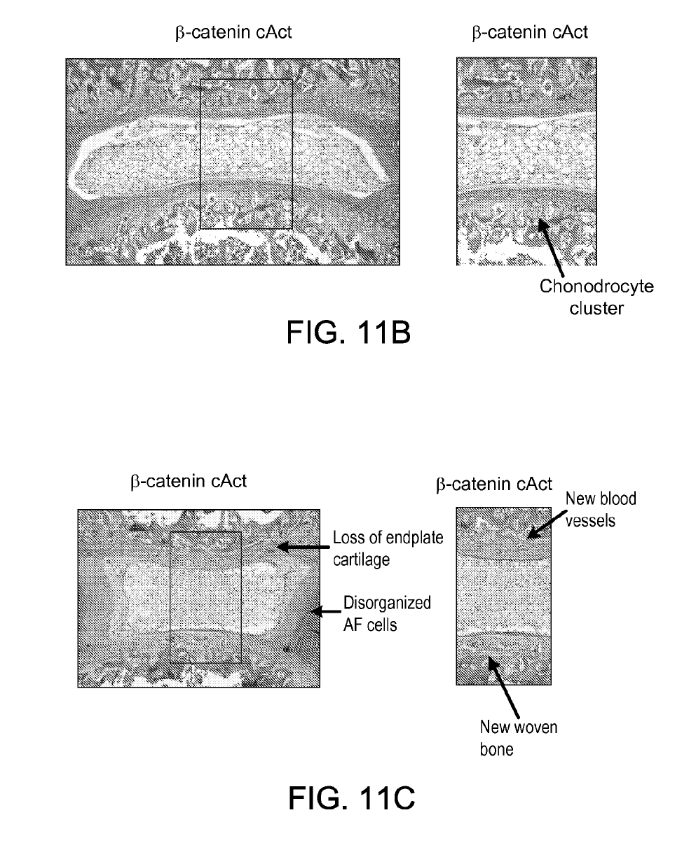

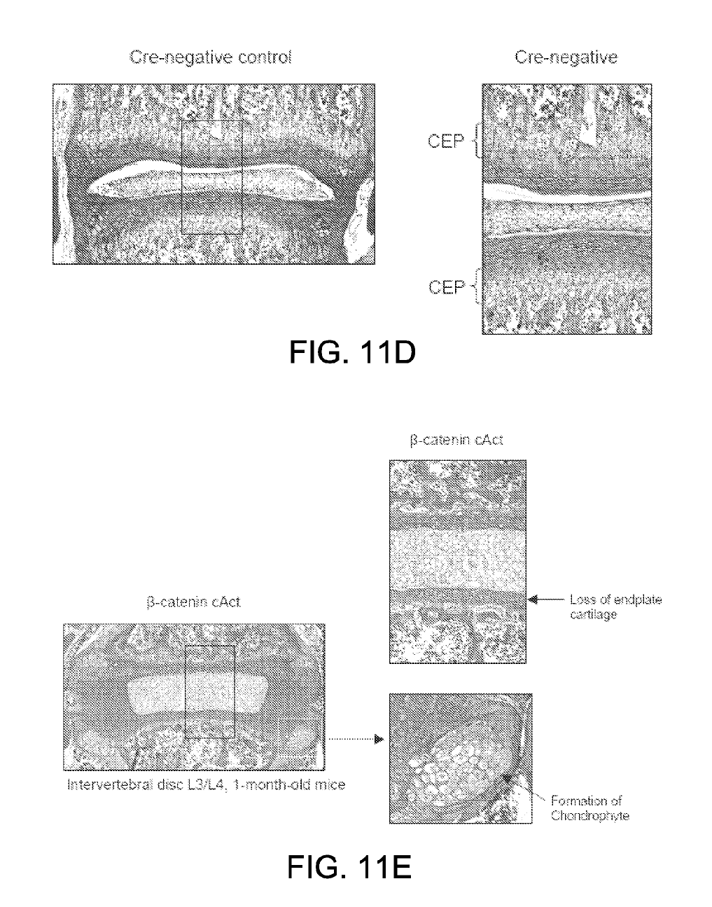

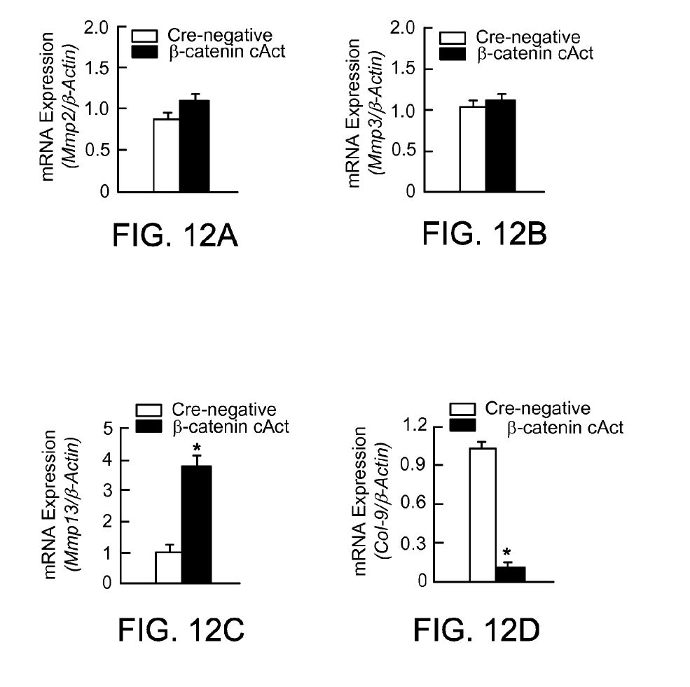

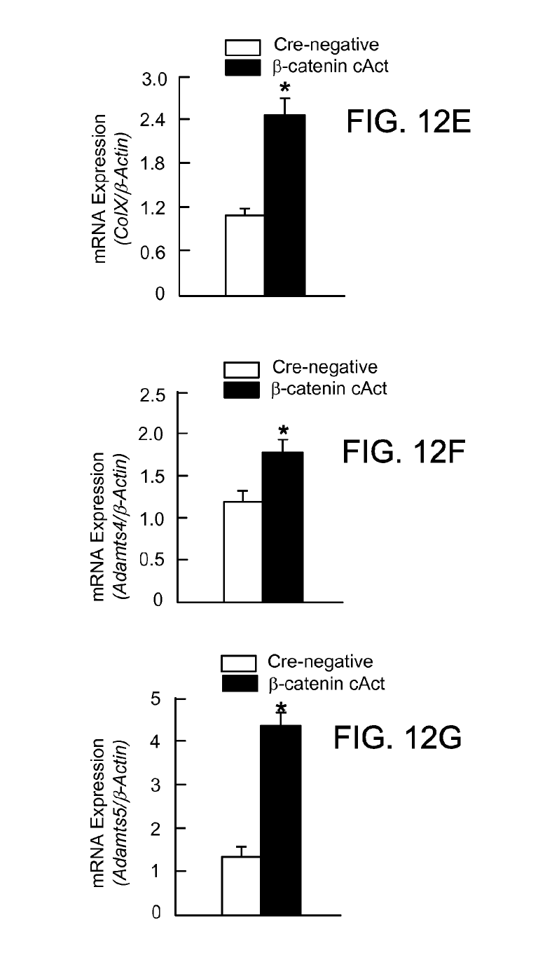

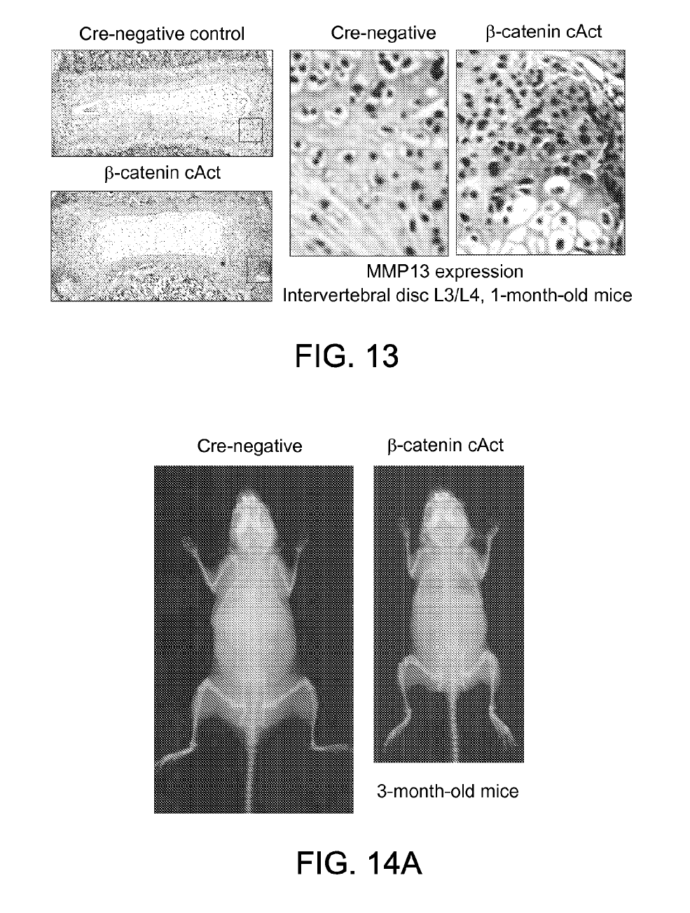

To characterize the IVD tissue phenotype of β-catenin cAct mice, micro-CT analysis was performed. Loss of endplate cartilage tissue was the major phenotype observed in β-catenin cAct mice (FIG. 10, lower panel). Some of the β-catenin cAct mice also showed disc space narrowing phenotype (FIG. 10, lower right panel). Detailed histological analysis showed that activation of β-catenin signaling in IVD tissue was associated with loss of endplate cartilage, formation of small chondrocyte clusters, and formation of new blood vessels and woven bones in the place where endplate cartilage should be (FIGS. 11B and 11C). In addition, disorganized annulus fibrosus cell morphology (FIG. 11C) and chondrophyte formation (FIG. 11E) were also found in β-catenin cAct mice. To determine changes in gene expression in disc cells, primary disc cells were isolated from 3-week-old β-catenin cAct mice and Cre-negative control mice and real-time PCR assays were performed. A 3-4 fold increase in mRNA expression of Mmp-13 and Adamts5 was found in disc cells derived from β-catenin cAct mice (FIGS. 12C and 12G). In contrast, no changes in expression of Mmp-2 and Mmp-3 and a small increase in Adamts4 expression were detected in β-catenin cAct mice (FIGS. 12A, 12B, and 12F). Col9 expression was dramatically suppressed and colX expression was significantly increased in disc cells of β-catenin cAct mice (FIGS. 12D and 12E). Consistent with finding on increased Mmp-13 mRNA expression, MMP13 protein levels were also significantly increased in IVD tissue of β-catenin cAct mice (FIG. 13). Fifteen 1-month-old β-catenin cAct mice were analyzed and the phenotypic changes observed in these mice were summarized in Table 4.

TABLE 4
Summary of IVD pneotype of 1-month-old β-catenin cAct mice.

| | Cre-Negative | β-catenin | |
|---|---|---|---|
| Phenotype | (n = 14) | cAct (n = 15) | |
| Reduced length of spine | 0 | 15 | (100%) |
| Disc space narrowing | 0 | 2 | (13%) |
| Loss of endplate cartilage | 0 | 15 | (100%) |
| New bone formation | 0 | 8 | (53%) |
| Chondrocyte formation | 0 | 10 | (67%) |
| Morphological changes of AF cells | 0 | 15 | (100%) |

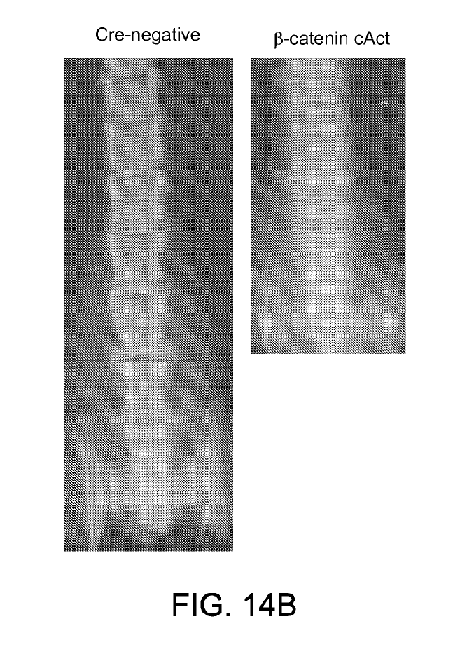

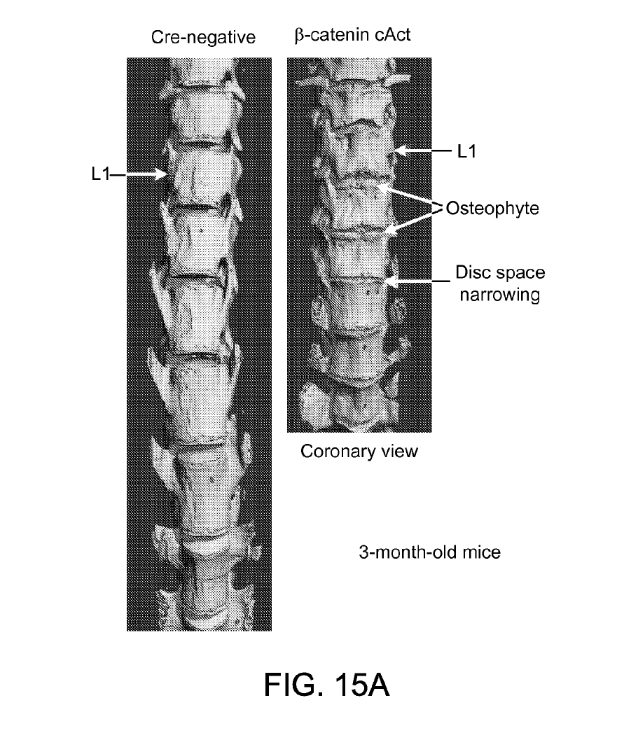

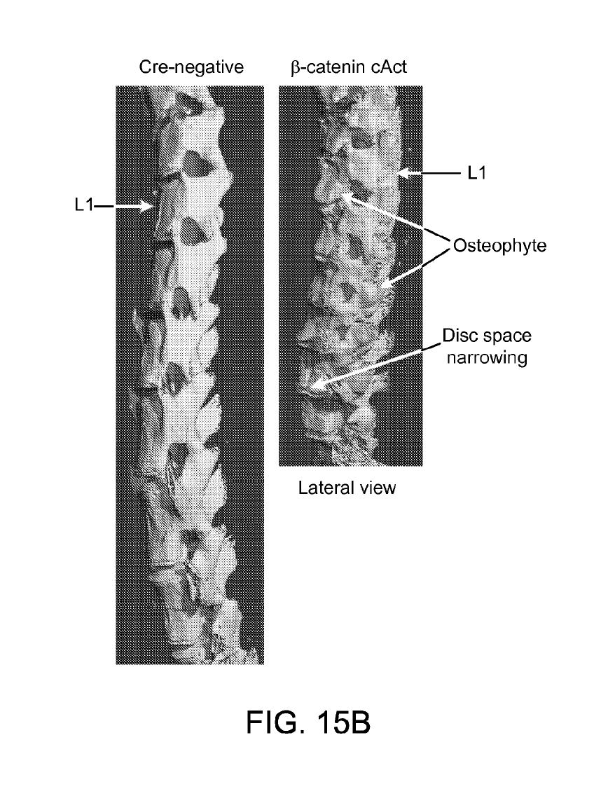

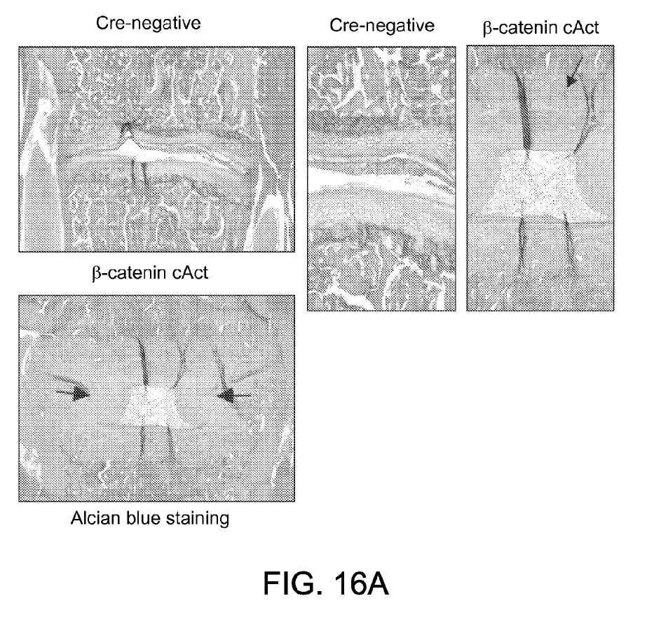

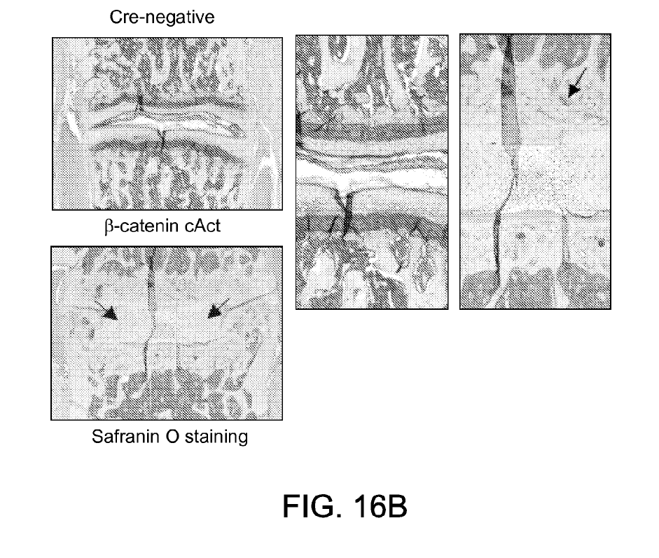

To further characterize phenotypic changes in older mice, 3-month-old β-catenin cAct mice were analyzed using X-ray, micro-CT and histological methods. X-ray radiographic analysis showed that over 20% reduction of the length of spine was observed in β-catenin cAct mice (FIG. 14). Massive amounts of osteophyte formation and disc space narrowing were also found in β-catenin cAct mice by micro-CT analysis (FIG. 15). Histological analysis showed that severe loss of proteoglycan protein and disorganized annulus fibrosus cell morphology were found in β-catenin cAct mice, demonstrated by Alcian blue and Safranin O staining (FIG. 16). Nine 3-month-old β-catenin cAct mice have been analyzed and several phenotypic changes, as shown in Table 5, were found in all of the β-catenin cAct mice, indicating the progression of the IVD tissue destruction with animal aging.

TABLE 5
Summary of IVD phenotype of 3-month-old β-catenin cAct mice.

| | Cre-Negative | β-catenin |
|---|---|---|
| Phenotype | (n= 14) | cAct (n = 15) |
| Reduced length of spine | 0 | 9 (100%) |
| Disc space narrowing | 0 | 9 (100%) |
| Loss of endplate cartilage | 0 | 9 (100%) |
| New bone formation | 0 | 9 (100%) |
| Chondrocyte formation | 0 | 9 (100%) |
| Morphological changes of AF cells | 0 | 9 (100%) |

### Example 8

### Mmp-13 Deletion Reverses β-Catenin cAct Phenotype

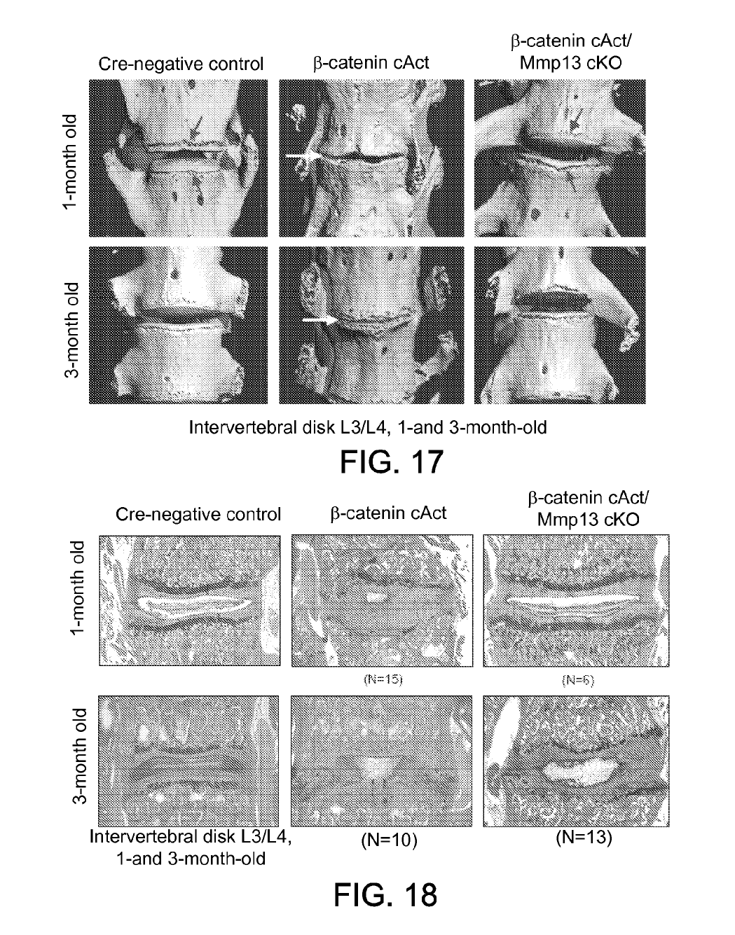

MMP13 plays a critical role in the development of osteoarthritis (Mitchell et al., J. Clin. Invest. 97:761-768 (1996); Neuhold et al., J. Clin. Invest. 107:35-44 (2001)). In the present studies, it has been discovered that Mmp-13 mRNA and protein were significantly increased in β-catenin cAct mice. To determine if Mmp-13 is a critical downstream target gene of β-catenin signaling, β-catenin cAct mice were bred with Mmp13^{fx/fx} mice and produced Col2a1-CreER^{T2};β-catenin^{fx(Ex3)/wt};Mmp13^{fx/fx} mice. In these mice, the cells, where the β-catenin signaling is activated and the Mmp-13 gene is deleted, were the same cell population because both the β-catenin and Mmp-13 genes are targeted by the Col2a1-CreER^{T2} transgenic mice. Micro-CT analysis showed that deletion of the Mmp-13 gene under the β-catenin cAct background significantly reversed the phenotypic changes in loss of endplate cartilage and disc space narrowing observed in β-catenin cAct mice (FIG. 17). Histological analysis further demonstrated that entire disc tissue morphology was returned to normal and proteoglycan protein levels were significantly increased and loss of endplate cartilage was restored when Mmp-13 gene was deleted under β-catenin cAct background in 1- and 3-month-old mice (FIG. 18).

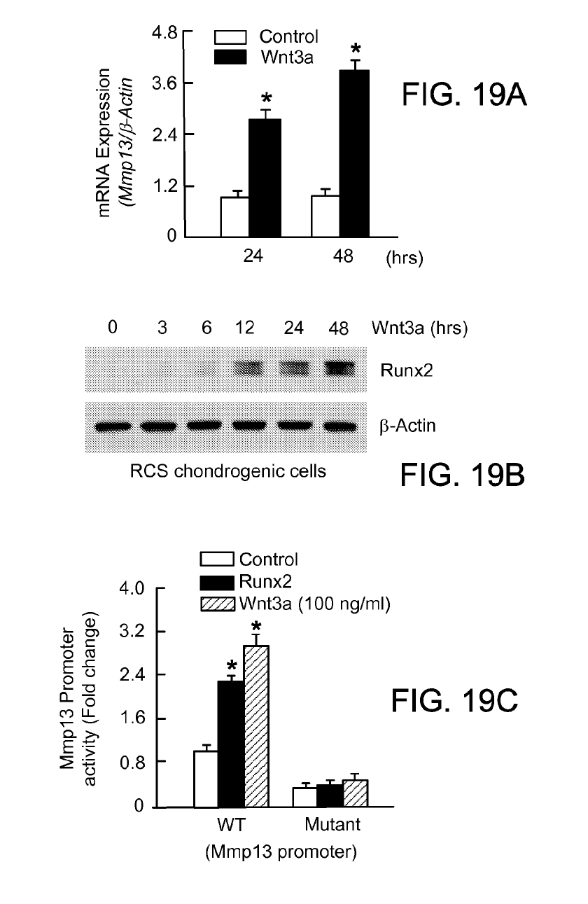

To determine the signaling mechanism through which β-catenin regulates Mmp-13 gene expression, in vitro studies using a RCS chondrogenic cell line were performed. Treatment with Wnt3a (canonical Wnt ligand) in RCS cells for 24 and 48 hours significantly increased Mmp-13 mRNA expression (FIG. 19A). Treatment with Wnt3a2 (0-48 hours) significantly up regulated Runx2 protein expression in a time-dependent manner (FIG. 19B). The 3.4 kb mouse Mmp-13 promoter was cloned and found that treatment with Wnt3a as well as transfection of Runx2 stimulated Mmp-13 promoter activity (FIG. 19C). A putative Runx2 binding site was identified within the 3.4 kb region of the Mmp-13 promoter. Mutation of this Runx2 binding site completely blocked the stimulatory effect of Runx2 as well as Wnt3a (FIG. 19C), suggesting that Wnt3a (or activation of β-catenin signaling) regulates Mmp-13 gene expression through up regulation of transcription factor Runx2.

1. A transgenic animal whose genome comprises:
(a) a first nucleic acid sequence encoding a fusion polypeptide, wherein the fusion polypeptide comprises a Cre recombinase and a mutated ligand binding domain of human estrogen receptor (CreER), wherein the first nucleic acid sequence is operably linked to a chondrocyte-specific promoter; and
(b) a second nucleic acid sequence encoding a β-catenin polypeptide, wherein the second nucleic acid sequence comprises one or more loxP sequences.

2. The transgenic animal of claim 1, wherein the chondrocyte-specific promoter is selected from the group consisting of a Col2a1 promoter, a fgfr-3 promoter, an aggrecan promoter, and a Col11a2 promoter.

3. (canceled)

4. The transgenic animal of 1, wherein the second nucleic acid sequence comprises two loxP sequences.

5. The transgenic animal of claim 4, wherein the second nucleic acid sequence further comprises at least a first exon, a second exon and a third exon.

6. The transgenic animal of claim 5, wherein a first loxP sequence is located 5′ to the third exon of the second nucleic acid sequence and a second loxP sequence is located 3′ to the third exon of the second nucleic acid sequence.

7. The transgenic animal of claim 1, wherein the first nucleic acid sequence comprises SEQ ID NO:1.

8. The transgenic animal of claim 1, wherein the second nucleic acid sequence comprises SEQ ID NO:2.

9-13. (canceled)

14. A progeny animal resulting from a cross between
(a) a first transgenic animal whose genome comprises a first nucleic acid sequence encoding a fusion polypeptide, wherein the fusion polypeptide comprises a Cre recombinase and a mutated ligand binding domain of human estrogen receptor (CreER), wherein the first nucleic acid sequence is operably linked to a chondrocyte-specific promoter; and
(b) a second transgenic animal whose genome comprises a second nucleic acid sequence encoding a β-catenin polypeptide, wherein the second nucleic acid sequence comprises one or more loxP sequences.

15. The progeny animal of claim 14, wherein the chondrocyte-specific promoter is selected from the group consisting of a Col2a1 promoter, a fgfr-3 promoter, an aggrecan promoter, and a Col11 a2 promoter.

16. (canceled)

17. The progeny animal of claim 14, wherein the second nucleotide sequence comprises two loxP sequences.

18. The progeny animal of claim 17, wherein the second nucleic acid sequence further comprises at least a first exon, a second exon and a third exon.

19. The progeny animal of claim 18, wherein a first loxP sequence is located 5′ to the third exon of the second nucleic acid sequence and a second loxP sequence is located 3′ to third exon of the second nucleic acid sequence.

20. The progeny animal of claim 14, wherein the first nucleic acid sequence comprises SEQ ID NO:l.

21. The progeny animal of claim 14, wherein the second nucleic acid sequence comprises SEQ ID NO:2.

22-26. (canceled)

27. A method of modifying the transgenic animal of claim 6 comprising administering tamoxifen to the transgenic animal, wherein administration of tamoxifen results in deletion of the third exon of the second nucleic acid sequence.

28. The method of claim 27, wherein deletion of the third exon of the second nucleic acid sequence results in a third nucleic acid sequence, wherein the third nucleic acid sequence encodes a β-catenin fusion polypeptide lacking the amino acids encoded by the third exon.

29. (canceled)

30. A modified transgenic animal made by the method of claim 27.

31. The modified transgenic animal of claim 30, wherein the third nucleic acid sequence comprises SEQ ID NO:3

32. An isolated cell of the modified transgenic animal of claim 30.

33. The isolated cell of claim 32, wherein the cell is selected from the group consisting of a chondrocyte, a fibroblast, and an intervertebral disc cell.

34. (canceled)

35. (canceled)

36. A method of screening for an agent that reduces or prevents one or more symptoms of osteoarthritis or intervertebral disc disease comprising the steps of:
(a) providing a transgenic animal of claim 30 whose genome comprises
(i) a first nucleic acid sequence encoding a fusion polypeptide, wherein the fusion polypeptide comprises a Cre recombinase and a mutated ligand binding domain of human estrogen receptor (CreER), wherein the first nucleic acid is operably linked to a chondrocyte-specific promoter; and
(ii) a second nucleic acid sequence encoding a β-catenin fusion polypeptide;
(b) administering to the transgenic animal an agent to be tested; and
(c) determining whether the agent reduces or prevents one or more symptoms of osteoarthritis or intervertebral disc disease.

37. The method of claim 36, wherein the determining step comprises determining the level of expression of the β-catenin fusion polypeptide or the level of RNA encoding the β-catenin fusion polypeptide, wherein a decrease in the level of expression of the β-catenin fusion polypeptide or the level of RNA encoding the β-catenin fusion polypeptide as compared to a control indicates the agent reduces or prevents one or more symptoms of osteoarthritis or intervertebral disc disease.

38-40. (canceled)

41. The method of claim 36, wherein the determining step includes determining the activity of the β-catenin fusion polypeptide, wherein a decrease in the activity of the β-catenin fusion polypeptide as compared to a control indicates the agent reduces or prevents osteoarthritis or intervertebral disc disease.

42. A method of screening for an agent that reduces or prevents one or more symptoms of osteoarthritis or intervertebral disc disease comprising the steps of:
(a) providing a transgenic animal whose genome comprises a first nucleic acid sequence comprising SEQ ID NO:1 and a second nucleic acid sequence comprising SEQ ID NO:3;
(b) administering to the transgenic animal an agent to be tested; and
(c) determining whether the agent reduces or prevents one or more symptoms of osteoarthritis or intervertebral disc disease.

43. A method of screening for an agent that reduces or prevents one or more symptoms of osteoarthritis or intervertebral disc disease comprising the steps of:
(a) providing a cell comprising
(i) a first nucleic acid sequence encoding a fusion polypeptide, wherein the fusion polypeptide comprises a Cre recombinase and a mutated ligand binding domain of human estrogen receptor (CreER), wherein the first nucleic acid is operably linked to a chondrocyte-specific promoter; and
(ii) a second nucleic acid sequence comprising a β-catenin fusion polypeptide;
(b) contacting the cell with an agent to be tested; and
(c) determining the level of expression or activity of the β-catenin fusion polypeptide in the cell, wherein a decrease in expression or activity of the β-catenin fusion polypeptide indicates the agent reduces or prevents one or more symptoms of osteoarthritis or intervertebral disc disease.

44. The method of claim 43, wherein the cell is isolated from a transgenic animal.

45-47. (canceled)

48. The method of claim 43, wherein the level of expression of RNA encoding the β-catenin fusion polypeptide or the level of expression of the β-catenin fusion polypeptide is determined.

49-51. (canceled)

52. The method of claim 43, wherein the level of activity of the β-catenin fusion polypeptide is determined.

53. A method of identifying a subject with or at risk for developing osteoarthritis or intervertebral disc disease comprising:
(a) obtaining a biological sample from the subject; and
(b) determining the level of expression or activity of β-catenin in the sample, wherein an increase inβ-catenin expression or activity as compared to a control indicates the subject has or is at risk for developing osteoarthritis or intervertebral disc disease.

54. The method of claim 53, wherein the biological sample comprises chondrocytes or fibroblasts.

55. (canceled)

56. The method of claim 53, wherein the level of RNA encoding β-catenin or the level of expression of β-catenin polypeptide is determined.

57-59. (canceled)

60. The method of claim 53, wherein the level of activity of the β-catenin is determined.

61. The method of claim 53, further comprising determining the level of expression or activity of one or more of aggrecan, Mmp-9, Mmp-13, alkaline phosphatase (Alp), osteocalcin (Oc), type X collagen (colX), Bmp2, Wnt5, Wnt11, sFRP2, WISP1, Adamts4, or Adamts5 wherein an increase in the level of expression or activity of aggrecan, Mmp-9, Mmp-13, Alp, Oc, colX, Bmp2, Wnt5, Wnt11, sFRP2, WISP1, Adamts4, or Adamts 5 indicates the subject has or is at risk for developing osteoarthritis or intervertebral disc disease.

62. The method of claim 53, further comprising determining the level of expression or activity of one or more of col9, Wnt1, Wnt3a, or Wnt7a, wherein a decrease in the level of expression or activity of col9, Wnt1, Wnt3a, or Wnt7a indicates the subject has or is at risk for developing osteroarthritis or intervertebral disc disease.

63. A method of treating or preventing osteoarthritis or intervertebral disc disease in a subject comprising:
(a) selecting a subject with or at risk of developing osteoarthritis or intervertebral disc disease; and
(b) administering to the subject an effective amount of a first therapeutic agent comprising a β-catenin inhibitor or a MMP-13 inhibitor.

64. The method of claim 63, wherein the subject has osteoarthritis and the first therapeutic agent comprises a β-catenin inhibitor.

65. (canceled)

66. The method of claim 64, wherein the β-catenin inhibitor is a nucleic acid molecule.

67. (canceled)

68. The method of claim 66, wherein the nucleic acid molecule is an siRNA molecule, wherein the siRNA molecule sequence targets SEQ ID NO:4 or SEQ ID NO:5.

69. (canceled)

70. (canceled)

71. The method of claim 64, wherein the β-catenin inhibitor is a polypeptide selected from the group consisting of an antibody, secreted frizzled-related protein 3 (sFRP3), and glycogen synthase kinase-3β (GSK-3β).

72-74. (canceled)

75. The method of claim 63, wherein the subject has intervertebral disc disease and the first therapeutic agent comprises a MMP-13 inhibitor.

76. (canceled)

77. The method of claim 75, wherein the MMP-13 inhibitor is a nucleic acid molecule.

78. (canceled)

79. The method of claim 77, wherein the nucleic acid molecule is an siRNA molecule, wherein the siRNA molecule sequence targets a sequence selected from the group consisting of SEQ ID NO:6, 7, 8, 9, or a combination thereof.

80. The method of claim 75, wherein the MMP-13 inhibitor is a small molecule selected from Wnt3a antagonist or Runx2 antagonist.

81-84. (canceled)

85. The method of claim 63, further comprising administering a second therapeutic agent to the subject.
